SEQUENCE LISTING (27 sequences)

SEQ ID NO: 1 - DNA, 1746 bp, Adenovirus type 5
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 2 - protein, 581 aa, Adenovirus type 5
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 3 - DNA, 1749 bp, Adenovirus type 2
atgaaacgcgccagaccgtctgaagacaccttcaaccccgtgtatccatatgacacagaaaccgggcctccaactgtgccctttcttacccctccatttgtttcacccaatggtttccaagaaagtccccctggagttctctctctacgcgtctccgaacctttggacacctcccacggcatgcttgcgcttaaaatgggcagcggtcttaccctagacaaggccggaaacctcacctcccaaaatgtaaccactgttactcagccacttaaaaaaacaaagtcaaacataagtttggacacctccgcaccacttacaattacctcaggcgccctaacagtggcaaccaccgctcctctgatagttactagcggcgctcttagcgtacagtcacaagccccactgaccgtgcaagactccaaactaagcattgctactaaagggcccattacagtgtcagatggaaagctagccctgcaaacatcagcccccctctctggcagtgacagcgacacccttactgtaactgcatcacccccgctaactactgccacgggtagcttgggcattaacatggaagatcctatttatgtaaataatggaaaaataggaattaaaataagcggtcctttgcaagtagcacaaaactccgatacactaacagtagttactggaccaggtgtcaccgttgaacaaaactcccttagaaccaaagttgcaggagctattggttatgattcatcaaacaacatggaaattaaaacgggcggtggcatgcgtataaataacaacttgttaattctagatgtggattacccatttgatgctcaaacaaaactacgtcttaaactggggcagggacccctgtatattaatgcatctcataacttggacataaactataacagaggcctatacctttttaatgcatcaaacaatactaaaaaactggaagttagcataaaaaaatccagtggactaaactttgataatactgccatagctataaatgcaggaaagggtctggagtttgatacaaacacatctgagtctccagatatcaacccaataaaaactaaaattggctctggcattgattacaatgaaaacggtgccatgattactaaacttggagcgggtttaagctttgacaactcaggggccattacaataggaaacaaaaatgatgacaaacttaccctgtggacaaccccagacccatctcctaactgcagaattcattcagataatgactgcaaatttactttggttcttacaaaatgtgggagtcaagtactagctactgtagctgctttggctgtatctggagatctttcatccatgacaggcaccgttgcaagtgttagtatattccttagatttgaccaaaacggtgttctaatggagaactcctcacttaaaaaacattactggaactttagaaatgggaactcaactaatgcaaatccatacacaaatgcagttggatttatgcctaaccttctagcctatccaaaaacccaaagtcaaactgctaaaaataacattgtcagtcaagtttacttgcatggtgataaaactaaacctatgatacttaccattacacttaatggcactagtgaatccacagaaactagcgaggtaagcacttactctatgtcttttacatggtcctgggaaagtggaaaatacaccactgaaacttttgctaccaactcttacaccttctcctacattgcccaggaataa

SEQ ID NO: 4 - protein, 582 aa, Adenovirus type 2
MKRARPSEDTFNPVYPYDTETGPPTVPFLTPPFVSPNGFQESPPGVLSLRVSEPLDTSHGMLALKMGSGLTLDKAGNLTSQNVTTVTQPLKKTKSNISLDTSAPLTITSGALTVATTAPLIVTSGALSVQSQAPLTVQDSKLSIATKGPITVSDGKLALQTSAPLSGSDSDTLTVTASPPLTTATGSLGINMEDPIYVNNGKIGIKISGPLQVAQNSDTLTVVTGPGVTVEQNSLRTKVAGAIGYDSSNNMEIKTGGGMRINNNLLILDVDYPFDAQTKLRLKLGQGPLYINASHNLDINYNRGLYLFNASNNTKKLEVSIKKSSGLNFDNTAIAINAGKGLEFDTNTSESPDINPIKTKIGSGIDYNENGAMITKLGAGLSFDNSGAITIGNKNDDKLTLWTTPDPSPNCRIHSDNDCKFTLVLTKCGSQVLATVAALAVSGDLSSMTGTVASVSIFLRFDQNGVLMENSSLKKHYWNFRNGNSTNANPYTNAVGFMPNLLAYPKTQSQTAKNNIVSQVYLHGDKTKPMILTITLNGTSESTETSEVSTYSMSFTWSWESGKYTTETFATNSYTFSYIAQE

SEQ ID NO: 5 - DNA, 972 bp, Adenovirus type 35
atgaccaagagagtccggctcagtgactccttcaaccctgtctacccctatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacgaataa

SEQ ID NO: 6 - protein, 323 aa, Adenovirus type 35
MTKRVRLSDSFNPVYPYEDESTSQHPFINPGFISPNGFTQSPDGVLTLKCLTPLTTTGGSLQLKVGGGLTVDDTDGTLQENIRATAPITKNNHSVELSIGNGLETQNNKLCAKLGNGLKFNNGDICIKDSINTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDE

SEQ ID NO: 7 - protein, 9 aa, Artificial Sequence
  Description of Artificial Sequence Synthetic Adenovirus 2 fiber consensus motif
KLGXGLXFX

SEQ ID NO: 8 - DNA, 1773 bp, Artificial Sequence
  Description of Artificial Sequence Synthetic nucleotide sequence of the gene encoding 5T35H fiber protein (Ad5-CMV5-GFP-5T35H; Bp position 28338 - 30110)
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacgaataa

SEQ ID NO: 9 - protein, 590 aa, Artificial Sequence
  Description of Artificial Sequence Synthetic amino acid sequence of 5T35H fiber (the tail and shaft derived from Ad5 and knob region obtained from Ad35)
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDE

SEQ ID NO: 10 - DNA, 1764 bp, Artificial Sequence
  Description of Artificial Sequence Synthetic nucleotide sequence of the gene encoding 5T3H fiber protein (Ad5-CMV5-GFP-5T3H; Bp position 28338 - 30097)
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacccctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgaataa

SEQ ID NO: 11 - protein, 587 aa, Artificial Sequence
  Description of Artificial Sequence Synthetic amino acid sequence of 5T3H fiber (the tail and shaft derived from Ad5 and the knob region obtained from Ad3)
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTGPKPEANCIIEYGKQNPDSKLTLILVKNGGIVNGYVTLMGASDYVNTLFKNKNVSINVELYFDATGHILPDSSSLKTDLELKYKQTADFSARGFMPSTTAYPFVLPNAGTHNENYIFGQCYYKASDGALFPLEVTVMLNKRLPDSRTSYVMTFLWSLNAGLAPETTQATLITSPFTFSYIREDE

SEQ ID NO: 12 - DNA, 1836 bp, Artificial Sequence
  Description of Artificial Sequence Synthetic nucleotide sequence of the gene encoding 5T3H-RGD fiber protein (Ad5-CMV5-GFP-5T3H-RGD; Bp position 30217 - 32052)
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaaccctatggacaggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgacggtggaggcggttcaggcggaggtggctctggcggtggcggatcctgtgactgccgcggagactgtttctgctaa

SEQ ID NO: 13 - protein, 611 aa, Artificial Sequence
  Description of Artificial Sequence Synthetic amino acid sequence of 5T3H-RGD fiber (the tail and shaft derived from Ad5 and the knob region obtained from Ad3)
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTGPKPEANCIIEYGKQNPDSKLTLILVKNGGIVNGYVTLMGASDYVNTLFKNKNVSINVELYFDATGHILPDSSSLKTDLELKYKQTADFSARGFMPSTTAYPFVLPNAGTHNENYIFGQCYYKASDGALFPLEVTVMLNKRLPDSRTSYVMTFLWSLNAGLAPETTQATLITSPFTFSYIREDDGGGGSGGGGSGGGGSCDCRGDCFC

SEQ ID NO: 14 - protein, 144 aa, Homo sapiens [sequence omitted]

SEQ ID NO: 15 - DNA, 2027 bp, Homo sapiens
tgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatggtaagtgagagaatgtgggcctgtgcctaggccacccagctggcccctgactggccacgcctgtcagcttgataacatgacattttccttttctacagaatgaaacagtagaagtcatctcagaaatgtttgacctccaggtaagatgcttctctctgacatagctttccagaagcccctgccctggggtggaggtggggactccattttagatggcaccacacagggttgtccactttctctccagtcagctggctgcaggaggagggggtagcaactgggtgctcaagaggctgctggccgtgcccctatggcagtcacatgagctcctttatcagctgagcggccatgggcagacctagcattcaatggccaggagtcaccaggggacaggtggtaaagtgggggtcacttcatgagacaggagctgtgggtttggggcgctcactgtgccccgagaccaagtcctgttgagacagtgctgactacagagaggcacagaggggtttcaggaacaacccttgcccacccagcaggtccaggtgaggccccacccccctctccctgaatgatggggtgagagtcacctccttccctaaggctgggctcctctccaggtgccgctgagggtggcctgggcggggcagtgagaagggcaggttcgtgcctgccatggacagggcagggtctatgactggacccagcctgtgcccctcccaagccctactcctgggggctgggggcagcagcaaaaaggagtggtggagagttcttgtaccactgtgggcacttggccactgctcaccgacgaacgacattttccacaggagccgacctgcctacagacccgcctggagctgtacaagcagggcctgcggggcagcctcaccaagctcaagggccccttgaccatgatggccagccactacaagcagcactgccctccaaccccggtgagtgcctacggcagggcctccagcaggaatgtcttaatctagggggtggggtcgacatggggagagatctatggctgtggctgttcaggaccccagggggtttctgtgccaacagttatgtaatgattagccctccagagaggaggcagacagcccatttcatcccaaggagtcagagccacagagcgctgaagcccacagtgctccccagcaggagctgctcctatcctggtcattattgtcattatggttaatgaggtcagaggtgagggcaaacccaaggaaacttggggcctgcccaaggcccagaggaagtgcccaggcccaagtgccaccttctggcaggactttcctctggccccacatggggtgcttgaattgcagaggatcaaggaaggggggctacttggaatggacaaggacctcaggcactccttcctgcgggaagggagcaaagtttgtggccttgactccactccttctgggtgcccagagacgacctcagcccagctgccctgctctgccctgggaccaaaaaggcaggcgtttgactgcccagaaggccaacctcaggctggcacttaagtcaggcccttgactctggctgccactggcagagctatgcactccttggggaacacgtgggtggcagcagcgtcacctgacccaggtcagtgggtgtgtcctggagtgggcctcctggcctctgagttctaagaggcagtagagaaacatgctggtgcttccttcccccacgttacccacttgcctggactcaagtgttttttatttttctttttttaaaggaaacttcctgtgcaacccagattatcacctttgaaagtttcaaagagaacctgaaggactttctgcttgtcatcccctttgactgctgggagccagtccaggagtg

SEQ ID NO: 16 - DNA, 435 bp, Homo sapiens
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtaa

SEQ ID NO: 17 - DNA, 44 bp, Artificial Sequence
  Description of Artificial Sequence Synthetic Adenovirus 5 PCR Primer 1
gaattctagggataacagggtaatcatcatcaataatatacctt

SEQ ID NO: 18 - DNA, 23 bp, Artificial Sequence
  Description of Artificial Sequence Synthetic Adenovirus 5 PCR Primer 2
cccggggtgctccacataaatct

SEQ ID NO: 19 - DNA, 44 bp, Artificial Sequence
  Description of Artificial Sequence Synthetic Adenovirus 5 PCR Primer 3
aagctttagggataacagggtaatcatcatcaataatatacctt

SEQ ID NO: 20 - DNA, 24 bp, Artificial Sequence
  Description of Artificial Sequence Synthetic Adenovirus 5 PCR Primer 4
cccgggggaatacatacccgcagg

SEQ ID NO: 21 - DNA, 37 bp, Artificial Sequence
  Description of Artificial Sequence Synthetic Human GM-CSF PCR primer PSR3
cttcgaggaattcaggatgtggctgcagagcctgctg

SEQ ID NO: 22 - DNA, 33 bp, Artificial Sequence
  Description of Artificial Sequence Synthetic Human GM-CSF PCR primer PSR4
cttcgagaagcttactcctggactggctcccag

SEQ ID NO: 23 - DNA, 34 bp, Artificial Sequence
  Description of Artificial Sequence Synthetic SV40 late poly(A) signal PCR primer PSR6
cttcgagaagcttcagacatgataagatacattg

SEQ ID NO: 24 - DNA, 35 bp, Artificial Sequence
  Description of Artificial Sequence Synthetic SV40 late poly(A) PCR primer PSR7
cttcgagggatcctaccacatttgtagaggtttac

SEQ ID NO: 25 - protein, 4 aa, Artificial Sequence
  Description of Artificial Sequence Synthetic peptide
RKKR

SEQ ID NO: 26 - protein, 4 aa, Artificial Sequence
  Description of Artificial Sequence Synthetic peptide
RKRR

SEQ ID NO: 27 - protein, 4 aa, Artificial Sequence
  Description of Artificial Sequence Synthetic peptide
RRRR

(19) United States
(12) Patent Application Publication
(10) Pub. No.: US 2006/0062764 A1
(43) Pub. Date: Mar. 23, 2006

(54) Fiber-modified adenoviral vectors for enhanced transduction of tumor cells

(76) Inventors: Seshidar-Reddy Police, Chester Springs, PA (US); Shanthi Ganesh, San Francisco, CA (US); DeChao Yu, Palo Alto, CA (US)

Correspondence Address: DLA PIPER RUDNICK GRAY CARY US LLP, 1200 Nineteenth Street, N.W., Washington, DC, 20036-2412, US

(21) Appl. No.: 11/201,384

(22) Filed: Aug. 11, 2005

Related U.S. Application Data
(60) Provisional application No. 60/604,009, filed on Aug. 25, 2004.

(51) Int. Cl. A61K 48/00; C12N 15/861

(52) U.S. Cl. 424/93.2; 435/456

(57) ABSTRACT

Adenoviral vectors which effectively transduce primary tumor cells are provided. The adenoviral vectors comprise a chimeric adenovirus fiber protein which includes at least a portion of a Subgroup C adenovirus fiber shaft and at least a portion of a Subgroup B adenovirus or serotype 37 adenovirus head, wherein the head region binds CD46.

### CROSS-REFERENCE TO RELATED APPLICATIONS

This application claims the priority benefit of U.S. Provisional Patent Application No. 60/604,009, filed Aug. 25, 2004, which is incorporated by reference in its entirety.

### BACKGROUND OF THE INVENTION

1. Field of the Invention

The present invention relates to adenoviral vectors which comprise a chimeric fiber protein and exhibit enhanced transduction of tumor cells, in particular, primary tumor cells, as compared to non-chimeric adenoviral vectors.

2. Background of the Technology

Gene therapy serves as a means to provide an exogenous nucleotide sequence to a cell and is the basis of some of the most innovative and potentially powerful disease-fighting tools. This approach holds great potential in treating not only cancer, but many other diseases as well, including cystic fibrosis, anemia, hemophilia, diabetes, Hungtington's disease, AIDS, abnormally high serum cholesterol levels, certain immune deficiencies, and many forms of cancer. Gene therapy generally relies upon a delivery vehicle, such as a viral vector in order to provide the exogenous sequence to a cell. Recombinant adenoviral vectors have shown some therapeutic efficacy against these diseases. For reviews, see Kim et al. (1996) Mol. Med. Today 12:519-527 and Smith et al. (1996) Gene Therapy 3:496-502.

There are two main types of adenoviral vectors, replication-incompetent (replication defective) and replication-competent. Replication defective vectors traditionally lack one or more genes essential for replication. These replication incompetent viruses are propagated on cells that complement the essential gene(s) which are lacking. Replication incompetent adenoviral vectors have been used extensively to transduce cells in vitro and in vivo and express various transgenes. Replication competent adenoviral vectors traditionally do not lack any of the adenovirus genes essential for replication and may be engineered to selectively replicate in certain cells.

The immune system plays a critical role in the pathogenesis of a wide variety of cancers. When cancers progress, it is widely believed that the immune system either fails to respond sufficiently or fails to respond appropriately, allowing cancer cells to grow. Currently, standard medical treatments for cancer including chemotherapy, surgery, radiation therapy and cellular therapy have clear limitations with regard to both efficacy and toxicity. To date, these approaches have met with varying degrees of success dependent upon the type of cancer, general health of the patient, stage of disease at the time of diagnosis, etc. Improved strategies that combine specific manipulation of the immune response to cancer in combination with standard medical treatments may provide a means for enhanced efficacy and decreased toxicity.

The use of autologous cancer cells as vaccines to augment anti-tumor immunity has been explored for some time (Oettgen et al., “The History of Cancer Immunotherapy”, In: Biologic Therapy of Cancer, Devita et al. (eds.) J. Lippincot Co., pp 87-199, 1991; Armstrong T D and Jaffee E M, Surg Oncol Clin N Am. 11(3):681-96, 2002 and Bodey B et al., Anticancer Res 20(4):2665-76, 2000).

Numerous cytokines have been shown to play a role in regulation of the immune response to tumors. For example, U.S. Pat. No. 5,098,702 describes use of combinations of TNF, IL-2 and IFN-beta in synergistically effective amounts to combat existing tumors. U.S. Pat. Nos. 5,078,996, 5,637,483 and 5,904,920 describe the use of GM-CSF for treatment of tumors. However, direct administration of cytokines for cancer therapy may not be practical, as they are often systemically toxic. (See, for example, Asher et al., J. Immunol. 146: 3227-3234, 1991 and Havell et al., J. Exp. Med. 167: 1067-1085, 1988.)

An expansion of this approach involves the use of genetically modified tumor cells which express cytokines locally at the vaccine site. Activity has been demonstrated in tumor models using a variety of immunomodulatory cytokines, including IL-4, IL-2, TNF-alpha, G-CSF, IL-7, IL-6 and GM-CSF, as described in Golumbeck P T et al., Science 254:13-716, 1991; Gansbacher B et al., J. Exp. Med. 172:1217-1224, 1990; Fearon E R et al., Cell 60:397-403, 1990; Gansbacher B et al., Cancer Res. 50:7820-25, 1990; Teng M et al., PNAS 88:3535-3539, 1991; Columbo M P et al., J. Exp. Med. 174:1291-1298, 1991; Aoki et al., Proc Natl Acad Sci USA. 89(9):3850-4, 1992; Porgador A, et al., Nat Immun. 13(2-3):113-30, 1994; Dranoff G et al., PNAS 90:3539-3543, 1993; Lee C T et al., Human Gene Therapy 8:187-193, 1997; Nagai E et al., Cancer Immunol. Immonther. 47:2-80, 1998 and Chang A et al., Human Gene Therapy 11:839-850, 2000, respectively.

Clinical trials employing GM-CSF-expressing autologous or allogeneic cellular vaccines (GVAX®) have commenced for treatment of prostate cancer, melanoma, lung cancer, pancreatic cancer, renal cancer, and multiple myeloma (Dummer R., Curr Opin Investig Drugs 2(6):844-8, 2001; Simons J et al., Cancer Res. 15;59(20):5160-8, 1999; Soiffer R et al., PNAS 95:13141-13146, 1998; Simons J et al., Cancer Res. 15; 57:1537-1546, 1997; Jaffee E et al., J. Clin Oncol. 19:145-156, 2001; and Salgia R et al., J. Clin Oncol. 21:624-630, 2003).

Although adenoviral vectors have been used to transduce tumor cells, the transduction of primary tumor cells has generally been inefficient. Therefore there remains a need for improved transduction efficiency of tumor cells, in particular, primary tumor cells.

### SUMMARY OF THE INVENTION

The present invention provides compositions and methods for expressing a heterologous nucleic acid in a tumor cell by transducing the tumor cell with an adenovirus which has a chimeric fiber protein wherein the chimeric fiber protein includes at least a portion of a Subgroup B adenovirus or serotype 37 adenovirus head and at least a portion of a Subgroup C adenovirus or bovine adenovirus fiber shaft. In one embodiment, the tumor cells are transduced in vitro, ex vivo or in vivo. In a preferred embodiment the tumor cells are primary tumor cells.

In practicing the invention, the adenovirus may be replication competent (i.e. oncolytic) or replication incompetent and may or may not encode a transgene.

Accordingly, in one aspect there is provided a method for selective cytolysis, i.e., killing a neoplastic cell in a cell population that comprises contacting an effective amount of a replication competent viral vector and/or viral particle of the invention with the cell population under conditions wherein the viral vector and/or particle selectively replicates in and kills the neoplastic cells. The cell population may be in an in vivo, in vitro or in an ex vivo setting.

In one aspect, the invention provides methods of expressing a transgene, e.g., a cytokine (such as GM-CSF) in a tumor cell. In a preferred aspect, the tumor cell is a primary tumor cell. In addition, methods of making a tumor vaccine are provided. Also, provided are methods of delivering a tumor vaccine of the invention to a mammal.

The invention further provides adenoviral particles, wherein a targeting ligand is included in a capsid protein of the particle. In a further embodiment, the capsid protein is a fiber protein and the ligand is in the C-terminus or HI loop of the fiber protein.

### BRIEF DESCRIPTION OF THE FIGURES

FIG. 1 provides a representation of wild type and exemplary chimeric fiber constructs. All modified fibers may be incorporated into adenoviral vectors (e.g. Ad5 based vectors with the E1 region deleted). The vectors based on Av1nBg express beta-galactosidase under the control of RSV promoter. The vectors based on Ad5-CMV5-GFP express green fluorescent protein under the control of CMV immediate early promoter. The source of shaft and knob regions for each vector is shown in the figure.

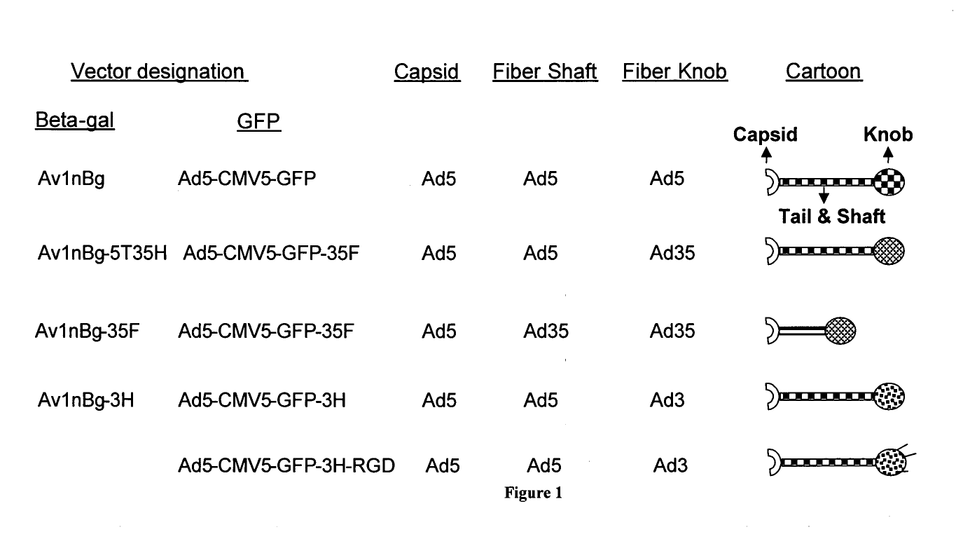

FIG. 2 is a graph illustrating the anti-tumor efficacy of OV1991 in the FaDu human head and neck tumor xenograft tumor model.

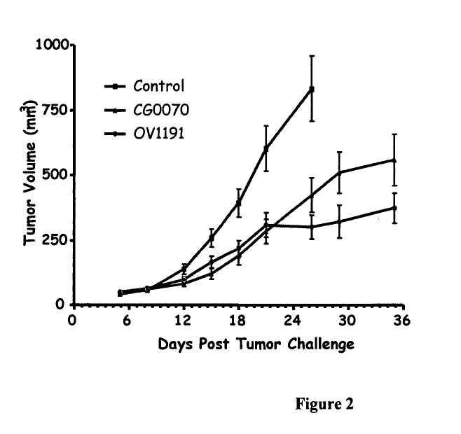

FIG. 3 is a graph illustrating the anti-tumor efficacy of fiber chimeric oncolytic vectors in the A375-luc human melanoma xenograft tumor model.

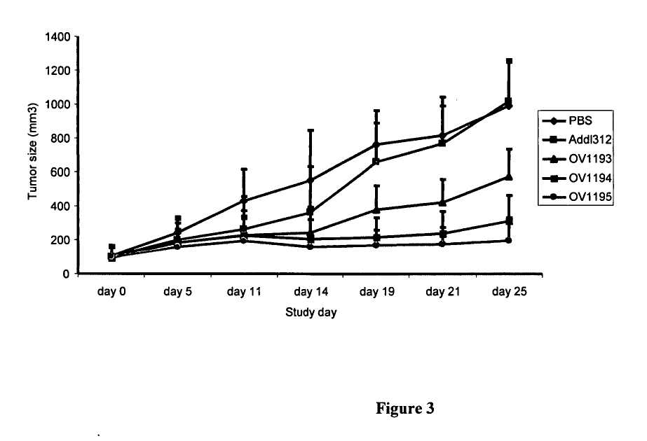

### DETAILED DESCRIPTION OF THE INVENTION

The present invention provides adenoviral compositions and methods for expressing a heterologous nucleic acid (e.g., a transgene) in a tumor cell by transducing the tumor cell with an adenovirus which has a chimeric fiber protein wherein the chimeric fiber protein comprises at least a portion of a Subgroup B adenovirus or serotype 37 adenovirus head and at least a portion of a Subgroup C adenovirus or bovine adenovirus fiber shaft. In one embodiment, the chimeric fiber protein comprises at least a portion of an Ad5 or Ad2 shaft and at least a portion of an Ad35 head. Typically the tumor cell is a primary tumor cell.

Without wishing to be bound by theory or mechanism, the present invention is based on the observation that adenoviral vectors comprising a chimeric fiber with an Ad5 or Ad2 shaft (or tail) region and an Ad35 head (knob) region more efficiently transduce certain cancer cells than those with an Ad2, Ad5 or Ad35 native fiber. Of particular interest is the ability of a chimeric adenovirus with a fiber shaft of a first serotype and a fiber head of a second serotype (e.g. an adenovirus having an Ad5 or Ad2 shaft (tail) and the head of a subgroup B adenovirus or serotype 37 adenovirus, to more efficiently transduce primary tumor cells than a non-chimeric adenoviruses of a single serotype. Nonlimiting examples of tumor cell types that are more efficiently transduced by the chimeric adenovirus vectors of the invention include lung tumor cells, prostate tumor cells, head and neck tumor cells, bladder tumor cells and kidney tumor cells. The transduction of tumor cells may be measured by expression of a marker gene (e.g., beta-galactosidase or GFP), however, any transgene may be included in and expressed from an adenovirus of the invention.

Ad5 is a subgroup C adenovirus and Ad35 is a subgroup B virus. The Ad35 fiber head region binds CD46 as do adenovirus serotypes 3 (Sirena et al., J Virol. May 2004; 78(9):4454-62), 11, 14, 16, 21, 35, 37 and 50. Ad37 belongs to adenovirus subgroup D. Preferred embodiments of the invention have the fiber shaft of a subgroup C adenovirus such as Ad2 or Ad5 and the fiber head of an adenovirus which binds CD46.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992, Current Protocols in Molecular Biology (John Wiley & Sons, including periodic updates); Glover, 1985, DNA Cloning (IRL Press, Oxford); Anand, 1992, Techniques for the Analysis of Complex Genomes, Academic Press, New York; Guthrie and Fink, 1991, Guide to Yeast Genetics and Molecular Biology, Academic Press, New York; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986).

### DEFINITIONS

Unless otherwise indicated, all terms used herein have the same meaning as they would to one skilled in the art and the practice of the present invention will employ, conventional techniques of microbiology and recombinant DNA technology, which are within the knowledge of those of skill of the art.

The publications and other materials including all patents, patent applications, publications (including published patent applications), and database accession numbers referred to in this specification are used herein to illuminate the background of the invention and in particular, cases to provide additional details respecting the practice. The publications and other materials including all patents, patent applications, publications (including published patent applications), and database accession numbers referred to in this specification are each incorporated herein by reference in its entirety.

In describing the present invention, the following terms are employed and are intended to be defined as indicated below.

The abbreviation “pfu” stands for plaque forming units.

The terms “virus,” “viral particle,” “vector particle,” “viral vector particle,” and “virion” are used interchangeably and are to be understood broadly as meaning infectious viral particles that are formed when, e.g., a viral vector of the invention is transduced into an appropriate cell or cell line for the generation of infectious particles. Viral particles according to the invention may be utilized for the purpose of transferring DNA into cells either in vitro or in vivo. For purposes of the present invention, these terms refer to adenoviruses, including recombinant adenoviruses formed when an adenoviral vector of the invention is encapsulated in an adenovirus capsid.

An “adenovirus vector” or “adenoviral vector” (used interchangeably) as referred to herein is a polynucleotide construct which can be packaged into an adenoviral virion. In some embodiments, an adenoviral vector of the invention includes a therapeutic gene sequence, e.g., a cytokine gene sequence. Exemplary adenoviral vectors of the invention include, but are not limited to, DNA, DNA encapsulated in an adenovirus coat, adenoviral DNA packaged in another viral or viral-like form (such as herpes simplex, and AAV), adenoviral DNA encapsulated in liposomes, adenoviral DNA complexed with polylysine, adenoviral DNA complexed with synthetic polycationic molecules, conjugated with transferrin, or complexed with compounds such as PEG to immunologically “mask” the antigenicity and/or increase half-life, or conjugated to a nonviral protein. Hence, the terms “adenovirus vector” or “adenoviral vector” as used herein include adenovirus or adenoviral particles.

The term as used herein “replication-competent” as used herein relative to the adenoviral vectors of the invention means the adenoviral vectors and particles of the invention preferentially replicate in certain types of cells or tissues but to a lesser degree or not at all in other types. In one embodiment of the invention, the adenoviral vector and/or particle selectively replicates in tumor cells and or abnormally proliferating tissue, such as solid tumors and other neoplasms. These include the viruses disclosed in U.S. Pat. Nos. 5,677,178, 5,698,443, 5,871,726, 5,801,029, 5,998,205, and 6,432,700 and PCT publications WO 95/19434, WO 98/39465, WO 98/39467, WO 98/39466, WO 99/06576, WO 98/39464, and WO 00/15820. Such viruses may be referred to as “oncolytic viruses” or “oncolytic vectors” and may be considered to be “cytolytic” or “cytopathic” and to effect “selective cytolysis” of target cells.

As used herein, the term “viral vector” is used according to its art-recognized meaning. It refers to a nucleic acid vector construct that includes at least one element of viral origin and may be packaged into a viral vector particle. The viral vector and/or particle may be utilized for the purpose of transferring DNA, RNA or other nucleic acids into cells either in vitro or in vivo. Numerous forms of viral vectors including adenoviral vectors are known in the art.

The term “derived from” when used in context to an adenoviral vector means the adenovirus serotype which is homologous to the adenoviral vector genome. In most cases, the majority of the adenoviral genome will be from one serotype and in this case the adenoviral vector is said to be derived from that one serotype. When the adenoviral vector genome has only one adenoviral coding sequences from a second serotype, this adenoviral vector is said to be derived from only the first adenovirus serotype.

The term “chimeric fiber protein” refers to an adenovirus fiber protein comprising a non-native amino acid sequence, in addition to or in place of a portion of a native fiber amino acid sequence. The non-native amino acid sequence may be from an adenoviral fiber protein of a different serotype. The non-native amino acid sequence may be any suitable length (e.g. 3 to about 200 amino acids). An exemplary “chimeric fiber protein” has a shaft derived from one adenoviral serotype and the head derived from a different adenoviral serotype.

The terms “vector,” “polynucleotide vector,” “polynucleotide vector construct,” “nucleic acid vector construct,” and “vector construct” are used interchangeably herein to mean any nucleic acid construct for gene transfer, as understood by one skilled in the art.

The term “gene essential for replication” refers to a nucleotide sequence whose transcription is required for a viral vector to replicate in a target cell. For example, in an adenoviral vector of the invention, a gene essential for replication may be selected from the group consisting of the E1a, E1b, E2a, E2b, and E4 genes.

As used herein, a “packaging cell” is a cell that is able to package adenoviral genomes or modified genomes to produce viral particles. It can provide a missing gene product or its equivalent. Thus, packaging cells can provide complementing functions for the genes deleted in an adenoviral genome and are able to package the adenoviral genomes into the adenovirus particle. The production of such particles requires that the genome be replicated and that those proteins necessary for assembling an infectious virus are produced. The particles also can require certain proteins necessary for the maturation of the viral particle. Such proteins can be provided by the vector or by the packaging cell.

The term “HeLa-S3” means the human cervical tumor-derived cell line available from American Type Culture Collection (ATCC, Manassas, Va.) and designated as ATCC number CCL-2.2. HeLa-S3 is a clonal derivative of the parent HeLa line (ATCC CCL-2). HeLa-S3 was cloned in 1955 by T. T. Puck et al. (J. Exp. Med 103: 273-284 (1956)).

A “self-processing cleavage site” or “self-processing cleavage sequence” as referred to herein is a DNA or amino acid sequence, wherein upon translation or as translated, rapid intramolecular (cis) cleavage of a polypeptide comprising the self-processing cleavage site occurs to result in expression of discrete mature protein or polypeptide products. Such a “self-processing cleavage site”, may also be referred to as a post-translational or co-translational processing cleavage site, e.g., a 2A site, sequence or domain. A 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly, 2001). Alternatively, a 2A site, sequence or domain demonstrates “auto-proteolysis” or “cleavage” by cleaving its own C-terminus in cis to produce primary cleavage products (Furler; Palmenberg, Ann. Rev. Microbiol. 44:603-623 (1990)).

The term “nucleic acid” refers to deoxyribonucleotides or ribonucleotides and polymers thereof (“polynucleotides”) in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid molecule/polynucleotide also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)). Nucleotides are indicated by their bases by the following standard abbreviations: adenine (A), cytosine (C), thymine (T), and guanine (G).

A nucleic acid is “operatively linked” when it is placed into a functional relationship with another nucleic acid. For example, a promoter or regulatory DNA sequence is said to be “operatively linked” to a DNA sequence that codes for an RNA and/or a protein if the two nucleotide sequences are operatively linked, or situated such that the promoter or regulatory DNA sequence affects the expression level of the coding or structural DNA sequence. Operatively linked DNA sequences are typically, but not necessarily, contiguous.

The terms “coding sequence” and “coding region” refer to a nucleotide sequence that is transcribed into RNA such as mRNA, rRNA, tRNA, snRNA, sense RNA or antisense RNA. In one embodiment, the RNA is then translated in a cell to produce a protein.

The term “ORF” means Open Reading Frame.

The term “gene” refers to a defined region that is located within a genome and that, in addition to the aforementioned coding sequence, comprises other, primarily regulatory, nucleotide sequences responsible for the control of expression, i.e., transcription and translation of the coding portion. A gene may also comprise other 5′ and 3′ untranslated sequences and termination sequences. Depending on the source of the gene, further elements that may be present are, for example, introns.

The terms “heterologous” and “exogenous” as used herein with reference to nucleic acid molecules such as promoters and gene coding sequences, refer to nucleotide sequences that originate from a source foreign to a particular virus or host cell or, if from the same source, are modified from their original form. Thus, a heterologous gene in a virus or cell includes a gene that is endogenous to the particular virus or cell but has been modified through, for example, codon optimization. The terms also include non-naturally occurring multiple copies of a naturally occurring nucleotide sequence. Thus, the terms refer to a nucleic acid segment that is foreign or heterologous to the virus or cell, or homologous to the virus or cell but in a position within the host viral or cellular genome in which it is not ordinarily found.

The term “homologous” as used herein with reference to a nucleic acid molecule refers to a nucleotide sequence naturally associated with a host virus or cell.

The terms “complement” and “complementary” refer to two nucleotide sequences that comprise antiparallel nucleotide sequences capable of pairing with one another upon formation of hydrogen bonds between the complementary base residues in the antiparallel nucleotide sequences.

The term “native” refers to a gene or protein that is present in the genome of the wildtype virus or cell.

The term “naturally occurring” or “wildtype” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

The term “targeting ligand” as used herein refers to a chemical moiety that preferentially directs an adenoviral particle to a desired cell type and/or tissue. The categories of such ligands include, but are not limited to, peptides, polypeptides, single chain antibodies, and multimeric proteins. Specific ligands include the TNF superfamily of ligands which include tumor necrosis factors (or TNF's) such as, for example, TNF-alpha. and TNF-beta., lymphotoxins (LT), such as LT-alpha. and LT-beta., Fas ligand which binds to Fas antigen; CD40 ligand, which binds to the CD40 receptor of B-lymphocytes; CD30 ligand, which binds to the CD30 receptor of neoplastic cells of Hodgkin's lymphoma; CD27 ligand, NGF ligand, and OX-40 ligand; transferrin, which binds to the transferrin receptor located on tumor cells, activated T-cells, and neural tissue cells; ApoB, which binds to the LDL receptor of liver cells; alpha-2-macroglobulin, which binds to the LRP receptor of liver cells; alpha-I acid glycoprotein, which binds to the asialoglycoprotein receptor of liver; mannose-containing peptides, which bind to the mannose receptor of macrophages; sialyl-Lewis-X antigen-containing peptides, which bind to the ELAM-I receptor of activated endothelial cells; CD34 ligand, which binds to the CD34 receptor of hematopoietic progenitor cells; ICAM-I, which binds to the LFA-I (CD11b/CD18) receptor of lymphocytes, or to the Mac-I (CD11a/CD18) receptor of macrophages; M-CSF, which binds to the c-fms receptor of spleen and bone marrow macrophages; circumsporozoite protein, which binds to hepatic Plasmodium falciparum receptor of liver cells; VLA-4, which binds to the VCAM-I receptor of activated endothelial cells; HIV gp120 and Class II MHC antigen, which bind to the CD4 receptor of T-helper cells; the LDL receptor binding region of the apolipoprotein E (ApoE) molecule; colony stimulating factor, or CSF, which binds to the CSF receptor; insulin-like growth factors, such as IGF-I and IGF-II, which bind to the IGF-I and IGF-II receptors, respectively; Interleukins 1 through 14, which bind to the Interleukin 1 through 14 receptors, respectively; the Fv antigen-binding domain of an immunoglobulin; gelatinase (MMP) inhibitor; bombesin, gastrin-releasing peptide; substance P; somatostatin; luteinizing hormone releasing hormone (LHRH); vasoactive peptide (VIP); gastrin; melanocyte stimulating hormone (MSH); cyclic RGD peptide and any other ligand or cell surface protein-binding (or targeting) molecule. In one embodiment of the invention, a peptide targeting ligand is inserted into a capsid protein of the adenoviral vector, typically the fiber protein of the adenoviral vector.

The term “recombinant” as used herein with reference to nucleic acid molecules refers to a combination of nucleic acid molecules that are joined together using recombinant DNA technology into a progeny nucleic acid molecule. As used herein with reference to viruses, cells, and organisms, the terms “recombinant,” “transformed,” and “transgenic” refer to a host virus, cell, or organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Recombinant viruses, cells, and organisms are understood to encompass not only the end product of a transformation process, but also recombinant progeny thereof. A “non-transformed,” “non-transgenic,” or “non-recombinant” host refers to a wildtype virus, cell, or organism that does not contain the heterologous nucleic acid molecule.

“Regulatory elements” are nucleotide sequences involved in controlling the expression of a nucleic acid. Regulatory elements include promoters, enhancers, and termination signals. They also typically encompass nucleotide sequences required for proper translation of the nucleotide sequence.

The term “promoter” refers to an untranslated DNA sequence usually located upstream of the coding region that contains the binding site for RNA polymerase II and initiates transcription of the DNA. The promoter region may also include other elements that act as regulators of gene expression. The term “minimal promoter” refers to a promoter element, particularly a TATA element that is inactive or has greatly reduced promoter activity in the absence of upstream activation elements.

The term “enhancer” within the meaning of the invention may be any genetic element, e.g., a nucleotide sequence that increases transcription of a coding sequence operatively linked to a promoter to an extent greater than the transcription activation effected by the promoter itself when operatively linked to the coding sequence, i.e. it increases transcription from the promoter.

The term “expression” refers to the transcription and/or translation of an endogenous gene, transgene or coding region in a cell. In the case of an antisense construct, expression may refer to the transcription of the antisense DNA only.

The term “up-regulated” as used herein means that a greater quantity of the RNA for a specific gene can be detected in the target cell as compared to another cell. For example, if a tumor cell that produces more telomerase RNA as compared to a non-tumor cell, the tumor cell has up-regulated expression of telomerase. Expression is considered up regulated when the quantity of specific RNA in a target cell (e.g. tumor cell) is at least 3-fold greater than in another cell (non-tumor cell). In another embodiment, the quantity of specific RNA is at least 5-fold greater. In another embodiment, the quantity of specific RNA is at least 10-fold greater. One skilled in the art knows how to measure RNA levels for a specific RNA sequence (e.g. Northern Assay).

The term “tumor selective promoter activity” as used herein means that the promoter activity of a promoter fragment of the present invention in tumor cells is higher than in non-tumor cell types.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. Jackson R J, Howell M T, Kaminski A (1990) Trends Biochem Sci 15(12):477-83) and Jackson R J and Kaminski, A. (1995) RNA 1(10):985-1000). The present invention encompasses the use of any IRES element, which is able to promote direct internal ribosome entry to the initiation codon of a cistron. “Under translational control of an IRES” as used herein means that translation is associated with the IRES and proceeds in a cap-independent manner. Examples of “IRES” known in the art include, but are not limited to IRES obtainable from picornavirus (Jackson et al., 1990, Trends Biochem Sci 15(12):477-483); and IRES obtainable from viral or cellular mRNA sources, such as for example, immunoglobulin heavy-chain binding protein (BiP), the vascular endothelial growth factor (VEGF) (Huez et al. (1998) Mol. Cell. Biol. 18(11):6178-6190), the fibroblast growth factor 2, and insulin-like growth factor, the translational initiation factor eIF4G, yeast transcription factors TFIID and HAP4. IRES have also been reported in different viruses such as cardiovirus, rhinovirus, aphthovirus, HCV, Friend murine leukemia virus (FrMLV) and Moloney murine leukemia virus (MoMLV). As used herein, “IRES” encompasses functional variations of IRES sequences as long as the variation is able to promote direct internal ribosome entry to the initiation codon of a cistron. In preferred embodiments, the IRES is mammalian. In other embodiments, the IRES is viral or protozoan. In one illustrative embodiment disclosed herein, the IRES is obtainable from encephelomycarditis virus (ECMV) (commercially available from Novogen, Duke et al. (1992) J. Virol 66(3):1602-1609). In another illustrative embodiment disclosed herein, the IRES is from VEGF. Examples of IRES sequences are described in U.S. Pat. No. 6,692,736.

The terms “identical” or percent “identity” in the context of two or more nucleotide or protein sequences, refer to two or more sequences or subsequences that are the same or have a specified percentage of amino acid residues or nucleotides that are the same, when compared and aligned for maximum correspondence, as measured using one of the sequence comparison algorithms described herein, e.g. the Smith-Waterman algorithm, or by visual inspection.

A “normal cell status” or “normal physiological state” is the status of a cell which exists in normal physiological conditions and which is non-dividing or divides in a regulated manner, i.e., a cell in a normal physiological state. An “aberrant cell status” is defined in relation to a cell of the same type, which is in a non-dividing/regulated dividing state and under normal physiological conditions. It follows that a cell which has an “aberrant cell status” exhibits unregulated cell division.

As used herein, the terms “cancer”, “cancer cells”, “neoplastic cells”, “neoplasia”, “tumor”, and “tumor cells” (used interchangeably) refer to cells that exhibit relatively autonomous growth, so that they exhibit an aberrant growth phenotype or aberrant cell status characterized by a significant loss of control of cell proliferation. A tumor cell may be a hyperplastic cell, a cell that shows a lack of contact inhibition of growth in vitro or in vivo, a cell that is incapable of metastasis in vivo, or a cell that is capable of metastasis in vivo. Neoplastic cells can be malignant or benign. It follows that cancer cells are considered to have an aberrant cell status.

The term “primary tumor cell” is used in accordance with the meaning in the art. A primary tumor cell is a cancer cell that is isolated from a tumor in a mammal and ahs not been extensively cultured in vitro.

The term “consists essentially of” or “consisting essentially of” as used herein with reference to a particular nucleotide sequence means that the particular nucleotide sequence may have additional residues on either the 5′ or 3′ end or both, wherein the additional residues do not materially affect the basic and novel characteristics of the recited sequence.

By the term “cytokine” or grammatical equivalents, herein is meant the general class of hormones of the cells of the immune system, including lymphokines, monokines, and others. The definition includes, without limitation, those hormones that act locally and do not circulate in the blood, and which, when used in accord with the present invention, will result in an alteration of an individual's immune response.

By the term “antigen from a tumor cell” or grammatical equivalents thereof, herein is meant any protein, carbohydrate or other component from a tumor cell capable of eliciting an immune response. The definition is meant to include, but is not limited to, using the whole tumor cell with all of its associated antigens as an antigen, as well as any component separated from the body of the cell, such as plasma membrane, cytoplasmic proteins, transmembrane proteins, proteins purified from the cell surface or membrane, or unique carbohydrate moieties associated with the cell surface. The definition also includes those antigens from the surface of the cell which require special treatment of the cells to access.

The term “genetically modified tumor cell” as used herein refers to a composition comprising a population of cells that has been genetically modified to express a transgene, and that is administered to a patient as part of a cancer treatment regimen. The genetically modified tumor cell vaccine comprises tumor cells which are “autologous” or “allogeneic” to the patient undergoing treatment or “bystander cells” that are mixed with tumor cells taken from the patient. A GM-CSF-expressing genetically modified tumor cell vaccine may be referred to herein as “GVAX”.

By the term “systemic immune response” or grammatical equivalents herein is meant an immune response which is not localized, but affects the individual as a whole, thus allowing specific subsequent responses to the same stimulus.

By the term “reversal of an established tumor” or grammatical equivalents herein is meant the suppression, regression, or partial or complete disappearance of a pre-existing tumor. The definition is meant to include any diminution in the size, potency or growth rate of a pre-existing tumor.

By the term “retarding the growth of a tumor” is meant the slowing of the growth rate of a tumor, the inhibition of an increase in tumor size or tumor cell number, or the reduction in tumor cell number, tumor size, or numbers of tumors.

By the term “transduction” is meant the introduction of an exogenous nucleic acid into a cell by physical means. For example, transduction includes the introduction of exogenous nucleic acid into a cell using a viral particle of the invention. For various techniques for manipulating mammalian cells, see Keown et al., Methods of Enzymology 185: 527-537 (1990).

The terms “treatment”, “therapeutic use”, or “medicinal use” as used herein, shall refer to any and all uses of the claimed compositions which remedy a disease state or symptoms, or otherwise prevent, hinder, retard, or reverse the progression of disease or other undesirable symptoms in any way whatsoever.

By the term “therapeutically effective amount” or grammatical equivalents herein refers to an amount of the preparation that is sufficient to modulate, either by stimulation or suppression, the systemic immune response of an individual. In the case of a cancer vaccine of the invention it is to stimulate the immune response of a mammal toward the cancer cells. This amount may be different for different individuals, different tumor types, and different preparations. The “therapeutically effective amount” is determined using procedures routinely employed by those of skill in the art such that an “improved therapeutic outcome” results.

As used herein, the terms “improved therapeutic outcome” and “enhanced therapeutic efficacy”, relative to cancer refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including an increase in life expectancy or an improvement in quality of life. By the terms “inactivated cells” and “proliferation-incompetent cells” or grammatical equivalents herein are meant cells inactivated by treatment rendering them proliferation-incompetent. This treatment results in cells which are unable to undergo multiple rounds of mitosis, but still retain the capability to express proteins such as cytokines and/or tumor antigens. This may be achieved through numerous methods known to those skilled in the art.

An “irradiated cell” is one example of such an inactivated cell. Such irradiated cells have been exposed to sufficient irradiation to render them proliferation-incompetent.

By the term “individual”, “subject” or grammatical equivalents thereof is meant any one individual mammal.

Target Cells

The present invention provides a method of expressing a heterologous nucleic acid in a target cell, such as a primary tumor cell. Target cells are transduced in vitro, in vivo or ex vivo with an adenovirus vector that has a chimeric fiber protein wherein the chimeric fiber protein comprises at least a portion of an Ad5 or Ad2 shaft and at least a portion of an Ad35 head.

The target cell may be of any cell or tissue type. In a preferred approach the target cell is tumor cell, typically a primary tumor cell. In one embodiment, the primary tumor cell is a cell selected from the group consisting of a lung tumor cell (e.g. a non-small cell lung tumor cell), a prostate tumor cell, a head and neck tumor cell, a bladder tumor cell, a melanoma tumor cell, a lymphoma cell and a kidney tumor cell.

In certain preferred embodiments, the primary tumor cell is a cancer cell of a type selected from the group consisting of bladder cancer, breast cancer, colon cancer, kidney cancer, liver cancer, lung cancer (e.g. non-small cell lung carcinoma), ovarian cancer, cervical cancer, pancreatic cancer, rectal cancer, prostate cancer, stomach cancer, epidermal cancer; a hematopoietic cancer of lymphoid or myeloid lineage; a cancer of mesenchymal origin such as a fibrosarcoma or rhabdomyosarcoma; other tumor types such as melanoma, teratocarcinoma, neuroblastoma, glioma and adenocarcinoma.

The advantage provided by the chimeric adenoviral vectors of the present invention is the increased transduction of tumor cells, in particular, primary tumor cells. An increased transduction efficiency means less adenoviral vector is needed. This results in a decrease in the amount of adenoviral vector that must be produced. Also, since the adenoviral vector transduction efficiency is more potent, smaller volumes of virus can be used to achieve the same transduction efficiency than typically achieved with a nonchimeric adenoviral vector.

The adenoviral vectors and methods of the invention have several utilities. For example, adenoviral vectors have been used for decades to introduce and express heterologous coding sequences in cells. The present invention provides adenoviral vectors and methods that more efficiently introduce and express heterologous coding sequences in primary tumor cells. This provides an efficient tool for studying the effects of various transgenes on primary tumor cells. The vectors and methods of the invention may also be used to test different therapeutic transgenes in tumor cells, in particular primary tumor cells. The adenoviral vectors and methods of the invention provide vectors and methods for therapeutic applications, e.g., treating cancer in a mammal. The methods of the invention may be used in vitro, in vivo and/or in an ex vivo context for transducing tumor cells.

### ADENOVIRAL VECTORS OF THE INVENTION

As used herein, the terms “adenovirus” and “adenoviral particle” are used to refer to any and all viruses that may be categorized as an adenovirus, including any adenovirus that infects a human or an animal, including all groups, subgroups, and serotypes. Thus, as used herein, “adenovirus” and “adenovirus particle” refer to the virus itself or derivatives thereof and cover all serotypes and subtypes and both naturally occurring and recombinant forms, except where indicated otherwise. Such adenoviruses may be wildtype or may be modified in various ways known in the art or as disclosed herein. Such modifications include modifications to the adenovirus genome that are packaged in the particle in order to make an infectious virus. Such modifications include deletions known in the art, such as deletions in one or more of the E1a, E1b, E2a, E2b, E3, or E4 coding regions. The terms also include replication-specific adenoviruses; that is, viruses that preferentially replicate in certain types of cells or tissues but to a lesser degree or not at all in other types. Such viruses are sometimes referred to as “cytolytic” or “cytopathic” viruses (or vectors), and, if they have such an effect on neoplastic cells, are referred to as “oncolytic” viruses (or vectors).

Adenoviral vectors of the present invention code for a chimeric fiber protein and adenovirus particles of the invention contain a chimeric fiber protein wherein said chimeric fiber protein comprises at least a portion of an Ad35 head and at least a portion of an Ad5 or Ad2 shaft. The portions of the retained Ad35 head and Ad5 or Ad2 shaft provide for efficient transduction of cancer cells. The chimeric adenoviral fiber proteins, useful in practicing the invention are further described below.

Adenoviral stocks that can be employed according to the invention include any adenovirus serotype. Adenovirus serotypes 1 through 51 are currently available from American Type Culture Collection (ATCC, Manassas, Va.), and the invention includes any other serotype of adenovirus available from any source. The adenoviruses that can be employed according to the invention may be of human or non-human origin, such as bovine, porcine, canine, simian, avian. For instance, an adenovirus can be of subgroup A (e.g., serotypes 12, 18, 31), subgroup B (e.g., serotypes 3, 7, 11, 14, 16, 21, 34, 35, 50), subgroup C (e.g., serotypes 1, 2, 5, 6), subgroup D (e.g., serotypes 8, 9, 10, 13, 15, 17, 19, 20, 22-30, 32, 33, 36-39, 42-47, 49, 51), subgroup E (serotype 4), subgroup F (serotype 40, 41), or any other adenoviral serotype. Throughout the specification reference is made to specific nucleotides in adenovirus type 5. One skilled in the art can determine the corresponding nucleotides in other serotypes and therefore construct similar adenoviral vectors in other adenovirus serotypes. In one preferred embodiment, the adenoviral nucleic acid backbone is derived from adenovirus serotype 2 (Ad2), 5 (Ad5) or 35 (Ad35), or a chimeric adenovirus backbone comprising a combination of a portion of adenovirus serotype 2(Ad2) or 5 (Ad5) with a portion of adenovirus serotype 35 (Ad35). Numerous examples of human and animal adenoviruses are available in the American Type Culture Collection, found e.g., at http://www.atcc.org/SearchCatalogs/CellBiology.cfm.

The DNA and protein sequences of adenovirus serotypes 2, 5, 35 (strain Holden) and 35 (strain 35p) can be found in GenBank under Accession numbers NC_{—}001405, AY339865, AY128640 and AY271307, respectively, all of which are incorporated by reference herein in their entirety. The protein sequence for the adenovirus serotype 11 and 14 fiber can be found in GenBank under Accession numbers BAD11205 and BAB83691, respectively, and the DNA sequence for the adenovirus serotype 3 fiber can be found in GenBank under Accession numbers X01998 and M12411, all of which are incorporated by reference herein in their entirety. The DNA sequence for Adenovirus serotypes 16 and 21 can be found in GenBank under accession numbers AB073632 and AB073222, respectively, both of which are incorporated by reference herein in their entirety. Along with the sequence information, the GenBank entries include useful details such as references, location of splicing signals, polyadenylation sites, TATA signals, introns, start and stop codons for each identified gene, protein sequence, cDNA for each gene, and a list of sequence variations that exist throughout the literature.

Adenoviral vectors are limited by the size of their genome (Bett et al, J Virol 67:5911 -5921, 1993). This in turn limits the amount of heterologous DNA that may be inserted into the vector and therefore limits the amount and or length of heterologous coding sequences that may be incorporated into the adenovirus genome. Therefore, replication incompetent viruses are generally capable of the largest heterologous DNA insertions, but are still limited by the size of the adenovirus genomic DNA and the amount of adenoviral DNA deleted.

In the context of adenoviral vectors, the term “5′” is used interchangeably with “upstream” and means in the direction of the left inverted terminal repeat (ITR). In the context of adenoviral vectors, the term “3′” is used interchangeably with “downstream” and means in the direction of the right ITR.

The adenoviral vectors of the invention include replication incompetent-and replication competent vectors. A replication-incompetent vector does not replicate, or does so at very low levels, in the target cell. In one aspect, a replication incompetent vector has at least one coding region in E1a, E1b, E2a, E2b or E4 inactivated, usually by deleting or mutating, part or all of the coding region. Methods for propagating these vectors are well known in the art.

In another aspect, the adenoviral vector is replication-competent. Replication-competent vectors are able to replicate in the target cell. Replication competent viruses include wild-type viruses and viruses engineered to replicate in the target cell. These include replication-specific viruses. Replication specific viruses are designed to replicate specifically or preferentially in one type of a cell as compared to another.

Both replication defective and replication-competent adenoviral vectors are being developed as therapeutic agents for treatment of cancer. These vectors may have deletions of at least one E3 coding region or in some cases retain a native E3 region. (See, e.g., WO 02/067861, WO 01/02540).

In the instance of adenoviral vectors that replicate selectively in target cells, specific attenuated replication-competent viral vectors have been developed for which selectively replication in cancer cells preferentially destroys those cells. Various cell-specific replication-competent adenovirus constructs, which preferentially replicate in (and thus destroy) certain cell types, are described in, for example, WO 95/19434, WO 96/17053, WO 98/39464, WO 98/39465, WO 98/39467, WO 98/39466, WO 99/06576, WO 99/25860, WO 00/15820, WO 00/46355, WO 02/067861, WO 02/06862, U.S. patent application publication US20010053352 and U.S. Pat. Nos. 5,698,443, 5,871,726, 5,998,205, and 6,432,700. Replication-competent adenovirus vectors have been designed to selectively replicate in tumor cells.

The target cell can be a certain cell type, tissue type or have a certain cell status. Examples of replication specific viruses that find utility in the compositions and methods of the present invention are further described in U.S. Pat. Nos. 5,998,205, 5,846,945, 5,801,029 and PCT publications WO 95/19434, WO 98/39465, WO 98/39467, WO 98/39466, WO 99/06576, WO 98/39464, and WO 00/15820.

The terms “replication conditional viruses”, “preferentially replicating viruses”, “specifically replicating viruses” and “selectively replicating viruses” are terms that are used interchangeably and are replication competent viral vectors and particles that preferentially replicate in certain types of cells or tissues but to a lesser degree or not at all in other types. In one embodiment of the invention, the viral vector and/or particle selectively replicates in tumor cells and or abnormally proliferating tissue, such as solid tumors and other neoplasms. Such viruses may be referred to as “oncolytic viruses” or “oncolytic vectors” and may be considered to be “cytolytic” or “cytopathic” and to effect “selective cytolysis” of target cells.

“Preferential replication” and “selective replication” and “specific replication” may be used interchangeably and mean that the virus replicates more in a target cell than in a non-target cell. The virus replicates at a higher rate in target cells than non target cells, e.g. at least about 3 fold higher, at least about 10-fold higher, at least about 50-fold higher, and in some instances at least about 100-fold, 400-fold, 500-fold, 1000-fold or even 1×10⁶ higher. In one embodiment, the virus replicates only in the target cells (that is, does not replicate at all or replicates at a very low level in non-target cells).

In another embodiment of the invention, the adenoviral vector comprises a deletion of part or all of the E1B 19-kDa region. In one embodiment, the adenoviral vector comprises all of the E1 coding sequences except for a complete or partial deletion of the E1b19 kDa coding sequence. The deletion causes the E1b 19 kDa protein to not be expressed or the protein is non-functional. The adenoviral E1B 19-kDa region refers to the genomic region of the adenovirus E1B gene encoding the E1B 19-kDa product. According to wild-type Ad5, the E1B 19-kDa region is a 261 bp region located between nucleotide (nt) 1714 and nt 2244. The E1B 19-kDa region has been described in, for example, Rao et al., Proc. Natl. Acad. Sci. USA, 89:7742-7746. The present invention encompasses deletion of part or all of the E1B 19-kDa region as well as embodiments wherein the E1B 19-kDa region is mutated, as long as the deletion or mutation lessens or eliminates the inhibition of apoptosis associated with E1B-19 kDa.

In one embodiment of the invention, the chimeric fiber protein comprises a heterologous amino acid sequence (e.g. a ligand) in said at least a portion of the head of an adenovirus which binds to CD46, e.g. Ad35. In some embodiments of the invention, the heterologous amino acid sequence is located in the HI-loop or at the C-ternminus of at least a portion of the head of an adenovirus which binds to CD46, e.g. Ad35.

In one embodiment of the invention, the adenovirus comprises a deletion of an adenoviral coding sequence essential for replication. In one embodiment, the replication defective adenovirus has at least one deletion in at least one of the following regions: E1a, E1b, E2a, E2b and E4. In one embodiment, the adenovirus is replication incompetent in the primary tumor cell.

In some embodiments of the invention, the adenoviral vector comprises a deletion of at least one adenoviral E3 coding sequence. In one embodiment, the at least one adenoviral E3 coding sequence is selected from the group consisting of the coding sequences for the 19K, 14.7K, 14.5K, 12.5K, 11.6K, 10.4K and 6.7K E3 proteins. In one embodiment, all of the E3 coding sequences are deleted and/or mutated so as the corresponding protein is not expressed or is nonfunctional. In one embodiment, the adenovirus comprises the 10.4K, 14.5K and 14.7K E3 coding sequences. In one embodiment, all of the native E3 coding sequences are retained in the vector.

In an embodiment of the invention, adenovirus vectors replicate preferentially in carcinoma cells, which replication preference is indicated by comparing the level of replication (e.g., cell killing and/or titer) in carcinoma target cells to the level of replication in non-target cells, e.g., normal or control cells. Comparison of the adenovirus titer in a target carcinoma cell to the titer in a non-target cell type provides a key indication that the overall replication preference is enhanced in target cells and/or that replication is depressed in non-target cells.

In one aspect of the present invention, the adenovirus vectors comprise an intergenic IRES element(s), which links the translation of two or more coding sequences. The linked coding region may be two adenoviral coding regions, two transgene coding regions or an adenoviral coding region and a transgene coding region.

Adenovirus vectors comprising an IRES linking two adenoviral coding regions are stable and in some embodiments provide better specificity than vectors not containing an IRES. Another advantage of an adenovirus vector comprising an intergenic IRES is that the use of an IRES rather than a second TRE may provide additional space in the vector for an additional gene(s) such as a therapeutic gene. Examples of adenoviral vectors comprising an IRES are described in U.S. Pat. No. 6,692,736. In one aspect of the invention, the viral vectors disclosed herein typically comprise at least one IRES within a multicistronic transcript, wherein production of the multicistronic transcript is regulated by a heterologous, target cell-specific TRE. For adenovirus vectors comprising a second adenoviral coding region under control of an IRES, it is preferred that the endogenous promoter of second the coding region under translational control of an IRES be deleted so that the endogenous promoter does not interfere with transcription of the second coding region. It is preferred that the second coding region be in frame with the IRES if the IRES contains an initiation codon. If an initiation codon, such as ATG, is present in the IRES, it is preferred that the initiation codon of the second coding sequence is removed and that the IRES and the second coding sequence are in frame. Alternatively, if the IRES does not contain an initiation codon or if the initiation codon is removed from the IRES, the initiation codon of the second coding region is used. In one embodiment, the adenovirus vectors comprise the adenovirus essential genes, E1A and E1B genes, under the transcriptional control of a heterologous TRE, and an IRES introduced between E1A and E1B. Thus, both E1A and E1B are under common transcriptional control, and translation of the E1B coding region is obtained by virtue of the presence of the IRES. In one embodiment, E1A has its endogenous promoter deleted. In another embodiment, E1A has an endogenous enhancer deleted and in yet an additional embodiment, E1A has its endogenous promoter deleted and an E1A enhancer deleted. In another embodiment, E1B has its endogenous promoter deleted. In yet further embodiments, E1B has a deletion of part or all of the 19-kDa region of E1B.

In a preferred embodiment for the oncolytic adenovirus platform, bicistronic or multicistronic cassettes containing a self processing cleavage sequence such as a 2A or 2A-like sequence comprise adenoviral early viral genes (E1A, E1B, E2, E3, and/and or E4) or genes expressed later in the viral life cycle (fiber, penton, and hexon).

In certain instances, it may be desirable to enhance the degree and/or rate of cytotoxic activity, due to, for example, the relatively refractory nature or particular aggressiveness of the cancerous target cell. An example of a viral gene that contributes to cytotoxicity includes, but is not limited to, the adenovirus death protein (ADP) gene. In another embodiment disclosed herein, the adenovirus comprises the adenovirus E1B gene which has a deletion in or of its endogenous promoter. In other embodiments disclosed herein, the 19-kDa region of E1B is deleted.

To provide enhanced cytotoxicity to target cells, one or more transgenes having a cytotoxic effect may be present in the vector. Additionally, or alternatively, an adenovirus gene that contributes to cytotoxicity and/or cell death, such as the adenovirus death protein (ADP) gene, can be included in the vector, optionally under the selective transcriptional control of a heterologous TRE and optionally under the translational control of an IRES or a self-processing cleavage sequence, such as a 2A or 2A-like sequence. This could be accomplished by coupling the target cell-specific cytotoxic activity with cell-specific expression of, a heterologous gene or transgene.

Exemplary adenoviral vectors of the invention include, but are not limited to, DNA, DNA encapsulated in an adenovirus coat, adenoviral DNA packaged in another viral or viral-like form (such as herpes simplex, and AAV), adenoviral DNA encapsulated in liposomes, adenoviral DNA complexed with polylysine, adenoviral DNA complexed with synthetic polycationic molecules, conjugated with transferrin, or complexed with compounds such as PEG to immunologically “mask” the antigenicity and/or increase half-life, or conjugated to a nonviral protein.

The adenoviral vector particle may also include further modifications to the fiber protein as described below. In one embodiment, the adenoviral vectors of the invention further comprise a targeting ligand included in a capsid protein of the particle. For examples of targeted adenoviruses, see for example, WO 00/67576, WO 99/39734, U.S. Pat. No. 6,683,170, U.S. Pat. No. 6,555,368, U.S. Pat. No. 5,922,315, U.S. Pat. No. 5,543,328, U.S. Pat. No. 5,770,442 and U.S. Pat. No. 5,846,782.

In addition, the adenoviral vectors of the present invention may also contain modifications to other viral capsid proteins. Examples of these mutations include, but are not limited to those described in U.S. Pat. Nos. 5,731,190, 6,127,525, and 5,922,315. Other modified adenoviruses are described in U.S. Pat. Nos. 6,057,155, 5,543,328 and 5,756,086.

An “E3 region” (used interchangeably with “E3”) is a term well understood in the art and means the region of the adenoviral genome that encodes the E3 gene products. The E3 region has been described in various publications, including, for example, Wold et al. (1995) Curr. Topics Microbiol. Immunol. 199:237-274. A “portion” of the E3 region means less than the entire E3 region, and as such includes polynucleotide deletions as well as polynucleotides encoding one or more polypeptide products of the E3 region.

Adenoviral constructs containing an E3 region can be generated wherein homologous recombination between an E3-containing adenoviral plasmid, for example, BHGE3 (Microbix Biosystems Inc., Toronto) and a non-E3-containing adenoviral plasmid, is carried out.

Alternatively, an adenoviral vector comprising an E3 region can be introduced into cells, for example 293 cells, along with an adenoviral construct or an adenoviral plasmid construct, where they can undergo homologous recombination to yield adenovirus containing an E3 region. In this case, the E3-containing adenoviral vector and the adenoviral construct or plasmid construct contain complementary regions of adenovirus, for example, one contains the left-hand and the other contains the right-hand region, with sufficient sequence overlap as to allow for homologous recombination.

Alternatively, an E3-containing adenoviral vector of the invention can be constructed using other conventional methods including standard recombinant methods (e.g., using restriction nucleases and/or PCR), chemical synthesis, or a combination of any of these. Further, deletions of portions of the E3 region can be created using standard techniques of molecular biology.

In some embodiments, the adenovirus death protein (ADP), encoded within the E3 region, is maintained in an adenovirus vector. The ADP gene, under control of the major late promoter (MLP), appears to code for a protein (ADP) that is important in expediting host cell lysis. Tollefson et al. (1996) J. Virol. 70(4):2296; Tollefson et al. (1992) J. Virol. 66(6):3633. Thus, adenoviral vectors containing the ADP gene may render the adenoviral vector more potent, making possible more effective treatment and/or a lower dosage requirement.

In one embodiment, the replication-competent vector comprises a gene essential for replication under transcriptional control of a selective TRE. In one embodiment, an adenovirus vector is a replication competent cancer-specific vector comprising E1B, wherein E1B has a deletion of part or all of the 19-kDa region. In embodiments of the invention, the adenoviral gene essential for replication is an early gene, e.g. one or more of E1A, E1B, E2a, E2b and E4. In a further embodiment, one or more additional TREs may be operatively linked to one or more adenoviral genes essential for replication or a transgene, e.g., a therapeutic gene.

An adenovirus vector may further include an additional heterologous TRE, which may or may not be operatively linked to the same gene(s) as the target cell-specific TRE. For example a TRE (such as a cell type-specific or cell status-specific TRE) may be juxtaposed to a second type of target-cell-specific TRE. “Juxtaposed” means a target cell-specific TRE and a second TRE transcriptionally control the same gene. For these embodiments, the target cell-specific TRE and the second TRE may be in any of a number of configurations, including, but not limited to, (a) next to each other (i.e., abutting); (b) both 5′ to the gene that is transcriptionally controlled (i.e., may have intervening sequences between them); (c) one TRE 5′ and the other TRE 3′ to the gene.

Transcriptional Regulatory Elements

Transcriptional regulatory elements (TREs), as well as methods for their identification, isolation, characterization, genetic manipulation and use for regulation of operatively linked coding sequences, are known in the art. A TRE can be derived from the transcriptional regulatory sequence of a single gene, sequences from different genes can be combined to produce a functional TRE, or a TRE can be synthetically generated (e.g. the CTP4 promoter).

A TRE can be tissue-specific, tumor-specific, developmental stage-specific, cell status specific, etc., depending on the type of cell present in the tissue or tumor. Such TREs are collectively referred to herein as tissue-specific or target cell-specific. As described in more detail below, a target cell-specific TRE can comprise any number of configurations, including, but not limited to, a target cell-specific promoter and target cell-specific enhancer; a heterologous promoter and a target cell-specific enhancer; a target cell-specific promoter and a heterologous enhancer; a heterologous promoter and a heterologous enhancer; and multimers of the foregoing. The promoter and enhancer components of a target cell-specific TRE may be in any orientation and/or distance from the coding sequence of interest, as long as the desired target cell-specific transcriptional activity is obtained.

Transcriptional activation can be measured in a number of ways known in the art (and described in more detail below), but is generally measured by detection and/or quantitation of mRNA or the protein product of the coding sequence under control of (i.e., operatively linked to) the target cell-specific TRE.

As further discussed herein, a target cell-specific TRE can be of varying lengths, and of varying sequence composition. A target cell-specific TRE is preferentially functional in a limited population (or type) of cells, e.g., prostate cells, liver cells, melanoma cells, etc. Accordingly, in some embodiments, the TRE used is preferentially functional in any of the following tissue types: prostate; liver; breast; urothelial (bladder); colon; lung; ovarian; pancreas; stomach; and uterine.

As is readily appreciated by one skilled in the art, a TRE is a polynucleotide sequence, and, as such, can exhibit function over a variety of sequence permutations. Methods of nucleotide substitution, addition, and deletion are known in the art, and readily available functional assays (such as the CAT or luciferase reporter gene assay) allow one of ordinary skill to determine whether a sequence variant exhibits requisite cell-specific transcription regulatory function. Hence, functionally preserved variants of TREs, comprising nucleic acid substitutions, additions, and/or deletions, can be used in the vectors disclosed herein. Accordingly, variant TREs retain function in the target cell but need not exhibit maximal function. In fact, maximal transcriptional activation activity of a TRE may not always be necessary to achieve a desired result, and the level of induction afforded by a fragment of a TRE may be sufficient for certain applications. For example, if used for treatment or palliation of a disease state, less-than-maximal responsiveness may be sufficient if, for example, the target cells are not especially virulent and/or the extent of disease is relatively confined.

Certain base modifications may result in enhanced expression levels and/or cell-specificity. For example, nucleic acid sequence deletions or additions within a TRE can move transcription regulatory protein binding sites closer or farther away from each other than they exist in their normal configuration, or rotate them so they are on opposite sides of the DNA helix, thereby altering spatial relationship among TRE-bound transcription factors, resulting in a decrease or increase in transcription, as is known in the art. Thus, while not wishing to be bound by theory, the present disclosure contemplates the possibility that certain modifications of a TRE will result in modulated expression levels as directed by the TRE, including enhanced cell-specificity. Achievement of enhanced expression levels may be especially desirable in the case of more aggressive forms of neoplastic growth, and/or when a more rapid and/or aggressive pattern of cell killing is warranted (for example, in an immunocompromised subject).

A TRE for use in the present vectors may or may not comprise a silencer. The presence of a silencer (i.e., a negative regulatory element known in the art) can assist in shutting off transcription (and thus replication) in non-target cells. Thus, presence of a silencer can confer enhanced cell-specific vector replication by more effectively preventing replication in non-target cells. Alternatively, lack of a silencer may stimulate replication in target cells, thus conferring enhanced target cell-specificity.

Transcriptional activity directed by a TRE (including both inhibition and enhancement) can be measured in a number of ways known in the art (and described in more detail below), but is generally measured by detection and/or quantitation of mRNA and/or of a protein product encoded by the sequence under control of (i.e., operatively linked to) a TRE.

As discussed herein, a TRE can be of varying lengths, and of varying sequence composition. The size of a heterologous TRE will be determined in part by the capacity of the viral vector, which in turn depends upon the contemplated form of the vector. Generally minimal sizes are preferred for TREs, as this provides potential room for insertion of other sequences which may be desirable, such as transgenes and/or additional regulatory sequences. In one embodiment, such an additional regulatory sequence is a self-processing cleavage sequences such as a 2A or 2A-like sequence.

By way of example, an adenoviral vector can be packaged with extra sequences totaling up to about 105% of the genome size, or approximately 1.8 kb, without requiring deletion of viral sequences. If non-essential sequences are removed from the adenovirus genome, an additional 4.6 kb of insert can be tolerated (i.e., for a total insertion capacity of about 6.4 kb).

In the case of replication-competent adenoviral vectors, in order to minimize non-specific replication, endogenous (adenovirus) TREs (i.e., the native E1A and/or E1B promoter) are preferably removed from the vector. Besides facilitating target cell-specific replication, removal of endogenous TREs also provides greater insert capacity in a vector, which is of special concern if an adenoviral vector is to be packaged within a virus particle. Even more importantly, deletion of endogenous TREs prevents the possibility of a recombination event whereby a heterologous TRE is deleted and the endogenous TRE assumes transcriptional control of its respective adenovirus coding sequences (thus allowing non-specific replication). In one embodiment, an adenoviral vector is constructed such that the endogenous transcription control sequences of one or more adenoviral genes are deleted and replaced by one or more heterologous TREs. However, endogenous TREs can be maintained in the adenovirus vector(s), provided that sufficient cell-specific replication preference is preserved. These embodiments are constructed by inserting heterologous TREs between an endogenous TRE and a gene coding segment required for replication. Requisite cell-specific replication preference is determined by conducting assays that compare replication of the adenovirus vector in a cell which allows function of the heterologous TREs with replication in a cell which does not.

In some embodiments, a TRE will increase replication of a vector in a target cell by at least about 2-fold, at least about 5-fold, at least about 10-fold, at least about 20-fold, at least about 50-fold, at least about 100-fold, at least about 200-fold, at least about 400- to about 500-fold, at least about 1000-fold, compared to basal levels of replication in the absence of a TRE. The acceptable differential can be determined empirically (by measurement of mRNA levels using, for example, RNA blot assays, RNase protection assays or other assays known in the art) and will depend upon the anticipated use of the vector and/or the desired result.

Adenoviral vectors directed at specific target cells can be generated using TREs that are preferentially functional in a target cell. In one embodiment of the present invention, a target cell-specific or cell status-specific, heterologous TRE is tumor cell-specific. A vector can comprise a single tumor cell-specific TRE or multiple heterologous TREs which are tumor cell-specific and functional in the same cell. In another embodiment, a vector comprises one or more heterologous TREs which are tumor cell-specific and additionally comprises one or more heterologous TREs which are tissue specific, whereby all TREs are functional in the same cell.

In one embodiment, for the oncolytic adenovirus platform, bicistronic or multicistronic cassettes containing a self processing cleavage sequence such as a 2A or 2A-like sequence comprise adenoviral early viral genes (E1A, E1B, E2, E3, and/and or E4) or genes expressed later in the viral life cycle (fiber, penton, and hexon).

In certain instances, it may be desirable to enhance the degree and/or rate of cytotoxic activity, due to, for example, the relatively refractory nature or particular aggressiveness of the cancerous target cell. An example of a viral gene that contributes to cytotoxicity includes, but is not limited to, the adenovirus death protein (ADP) gene. In another embodiment disclosed herein, the adenovirus comprises the adenovirus E1B gene which has a deletion in or of its endogenous promoter. In other embodiments disclosed herein, the 19-kDa coding region of E1B contains a partial or complete deletion, so as the 19 kDa protein is not expressed or is non-functional.

To provide enhanced cytotoxicity to target cells, one or more transgenes having a cytotoxic effect may be present in the vector. Additionally, or alternatively, an adenovirus gene that contributes to cytotoxicity and/or cell death, such as the adenovirus death protein (ADP) gene, can be included in the vector, optionally under the selective transcriptional control of a heterologous TRE and optionally under the translational control of an IRES or a self-processing cleavage sequence, such as a 2A or 2A-like sequence. This could be accomplished by coupling the target cell-specific cytotoxic activity with cell-specific expression of, a heterologous gene or transgene.

Any of a number of heterologous therapeutic genes or transgenes may be included in the replication competent viral vectors of the invention, as further described below.

Typically, the aforementioned bicistronic or multicistronic cassettes are placed under the control of a transcriptional response element, generally a cell type or cell status associated transcriptional regulatory element that is preferentially expressed in cancer or tumor cells. Accordingly, the therapeutic gene included in a given construct will vary dependent upon the type of cancer under treatment.

As is known in the art, activity of TREs can be inducible. Inducible TREs generally exhibit low activity in the absence of inducer, and are up-regulated in the presence of an inducer. Inducers include, for example, nucleic acids, polypeptides, small molecules, organic compounds and/or environmental conditions such as temperature, pressure or hypoxia. Inducible TREs may be preferred when expression is desired only at certain times or at certain locations, or when it is desirable to titrate the level of expression using an inducing agent. For example, transcriptional activity from the PSE-TRE, PB-TRE and hKLK2-TRE is inducible by androgen, as described herein and in PCT/US98/04080, expressly incorporated by reference herein. Accordingly, in one embodiment of the present invention, the adenovirus vector comprises an inducible heterologous TRE.

A TRE as used in the present invention can be present in a variety of configurations. A TRE can comprise multimers. For example, a TRE can comprise a tandem series of at least two, at least three, at least four, or at least five target cell-specific TREs. These multimers may also contain heterologous promoter and/or enhancer sequences. Alternatively, a TRE can comprise one or more promoter regions along with one or more enhancer regions. TRE multimers can also comprise promoter and/or enhancer sequences from different genes. The promoter and enhancer components of a TRE can be in any orientation with respect to each other and can be in any orientation and/or any distance from the coding sequence of interest, as long as the desired cell-specific transcriptional activity is obtained.

In one embodiment, the adenovirus replicates preferentially in the tumor cells. In one embodiment, the adenovirus comprises a heterologous transcriptional regulatory element (TRE) operatively linked to at least one adenoviral coding sequence that is essential for replication. TREs include, but are not limited to a cell-specific TRE, a cell-status specific TRE and a tissue-specific TRE. Examples of specific TREs that may be used in the present invention include, but are not limited to, a PSA TRE, an E2F TRE, a telomerase (TERT) TRE, urokinase plasminogen activator (uPA) TRE, urokinase plasminogen activator receptor (uPAR) TRE, PRL-3 protein tyrosine phosphatase TRE. The invention also contemplates the use of combinations of TREs. An adenoviral coding sequence essential for replication may be operatively linked to more than one heterologous TRE. Alternatively or in addition, more than one adenoviral coding sequence essential for replication may be operatively linked to one or more heterologous TREs. Examples of adenoviral coding sequences essential for replication are the coding sequences located in the E1a, E1b, E2a, E2b and E4 regions. Multiple coding regions essential for replication may be operatively linked to one TRE or combinations of TREs, for example with an IRES. In one embodiment, at least one heterologous TRE is operatively linked to a first adenoviral coding sequence that is essential for replication, wherein the first adenoviral coding sequence is also operatively linked to an IRES and said IRES is also linked to a second adenoviral coding sequence essential for replication. For example, at least one heterologous TRE is operatively linked to the coding sequences of the E1a coding region and these E1a coding sequences also operatively linked to a downstream IRES, wherein the IRES is also operatively linked to the coding sequences of the E1b region.

As used herein, a TRE derived from a specific gene is referred to by the gene from which it was derived and is a polynucleotide sequence which regulates transcription of an operatively linked polynucleotide sequence in a host cell that expresses the gene. For example, as used herein, a “human glandular kallikrein transcriptional regulatory element”, or “hKLK2-TRE” is a polynucleotide sequence, preferably a DNA sequence, which increases transcription of an operatively linked polynucleotide sequence in a host cell that allows an hKLK2-TRE to function, such as a cell (preferably a mammalian cell, even more preferably a human cell) that expresses androgen receptor, such as a prostate cell. An hKLK2-TRE is thus responsive to the binding of androgen receptor and comprises at least a portion of an hKLK2 promoter and/or an hKLK2 enhancer (i.e., the ARE or androgen receptor binding site). Human glandular kallikrein enhancers and adenoviral vectors comprising the enhancer are described in WO99/06576, expressly incorporated by reference herein.

As used herein, a “probasin (PB) transcriptional regulatory element”, or “PB-TRE” is a polynucleotide sequence, preferably a DNA sequence, which selectively increases transcription of an operatively-linked polynucleotide sequence in a host cell that allows a PB-TRE to function, such as a cell (preferably a mammalian cell, more preferably a human cell, even more preferably a prostate cell) that expresses androgen receptor. A PB-TRE is thus responsive to the binding of androgen receptor and comprises at least a portion of a PB promoter and/or a PB enhancer (i.e., the ARE or androgen receptor binding site). Adenovirus vectors specific for cells expressing androgen are described in WO98/39466, expressly incorporated by reference herein.

As used herein, a “prostate-specific antigen (PSA) transcriptional regulatory element”, or “PSA-TRE”, or “PSE-TRE” is a polynucleotide sequence, preferably a DNA sequence, which selectively increases transcription of an operatively linked polynucleotide sequence in a host cell that allows a PSA-TRE to function, such as a cell (preferably a mammalian cell, more preferably a human cell, even more preferably a prostate cell) that expresses androgen receptor. A PSA-TRE is thus responsive to the binding of androgen receptor and comprises at least a portion of a PSA promoter and/or a PSA enhancer (i.e., the ARE or androgen receptor binding site). A tissue-specific enhancer active in prostate and used in adenoviral vectors is described in WO95/19434 and WO97/01358, each of which is expressly incorporated by reference herein.

As used herein, a “carcinoembryonic antigen (CEA) transcriptional regulatory element”, or “CEA-TRE” is a polynucleotide sequence, preferably a DNA sequence, which selectively increases transcription of an operatively linked polynucleotide sequence in a host cell that allows a CEA-TRE to function, such as a cell (preferably a mammalian cell, even more preferably a human cell) that expresses CEA. The CEA-TRE is responsive to transcription factors and/or co-factor(s) associated with CEA-producing cells and comprises at least a portion of the CEA promoter and/or enhancer. Adenovirus vectors specific for cells expressing carcinoembryonic antigen are described in WO98/39467, expressly incorporated by reference herein.

As used herein, an “alpha-fetoprotein (AFP) transcriptional regulatory element”, or “AFP-TRE” is a polynucleotide sequence, preferably a DNA sequence, which selectively increases transcription (of an operatively linked polynucleotide sequence) in a host cell that allows an AFP-TRE to function, such as a cell (preferably a mammalian cell, even more preferably a human cell) that expresses AFP. The AFP-TRE is responsive to transcription factors and/or co-factor(s) associated with AFP-producing cells and comprises at least a portion of the AFP promoter and/or enhancer. Adenovirus vectors specific for cells expressing alpha-fetoprotein are described in WO98/39465, expressly incorporated by reference herein.

As used herein, “a mucin gene (MUC) transcriptional regulatory element”, or “MUC1-TRE” is a polynucleotide sequence, preferably a DNA sequence, which selectively increases transcription (of an operatively-linked polynucleotide sequence) in a host cell that allows a MUC1-TRE to function, such as a cell (preferably a mammalian cell, even more preferably a human cell) that expresses MUC1. The MUC1-TRE is responsive to transcription factors and/or co-factor(s) associated with MUC1-producing cells and comprises at least a portion of the MUC1 promoter and/or enhancer.

As used herein, a “urothelial cell-specific transcriptional response element”, or “urothelial cell-specific TRE” is polynucleotide sequence, preferably a DNA sequence, which increases transcription of an operatively linked polynucleotide sequence in a host cell that allows a urothelial-specific TRE to function, i.e., a target cell. A variety of urothelial cell-specific TREs are known, are responsive to cellular proteins (transcription factors and/or co-factor(s)) associated with urothelial cells, and comprise at least a portion of a urothelial-specific promoter and/or a urothelial-specific enhancer. Exemplary urothelial cell specific transcriptional regulatory sequences include a human or rodent uroplakin (UP), e.g., UPI, UPII, UPIII and the like. Human urothelial cell specific uroplakin transcriptional regulatory sequences and adenoviral vectors comprising the same are described in WO01/72994, expressly incorporated by reference herein.

As used herein, a “melanocyte cell-specific transcriptional response element”, or “melanocyte cell-specific TRE” is a polynucleotide sequence, preferably a DNA sequence, which increases transcription of an operatively linked polynucleotide sequence in a host cell that allows a melanocyte-specific TRE to function, i.e., a target cell. A variety of melanocyte cell-specific TREs are known, are responsive to cellular proteins (transcription factors and/or co-factor(s)) associated with melanocyte cells, and comprise at least a portion of a melanocyte-specific promoter and/or a melanocyte-specific enhancer. Methods are described herein for measuring the activity of a melanocyte cell-specific TRE and thus for determining whether a given cell allows a melanocyte cell-specific TRE to function. Examples of a melanocyte-specific TRE for use in practicing the invention include but are not limited to a TRE derived from the 5′ flanking region of a tyrosinase gene, a tyrosinase related protein-1 gene, a TRE derived from the 5′-flanking region of a tyrosinase related protein-2 gene, a TRE derived from the 5′ flanking region of a MART-1 gene or a TRE derived from the 5′-flanking region of a gene which is aberrantly expressed in melanoma.

In another aspect, the invention provides adenoviral vectors comprising a metastatic colon cancer specific TRE derived from a PRL-3 gene operatively linked to a gene essential for adenovirus replication or a transgene. As used herein, a “metastatic colon cancer specific TRE derived from a PRL-3 gene” or a “PRL-3 TRE” is a polynucleotide sequence, preferably a DNA sequence, which selectively increases transcription of an operatively linked polynucleotide sequence in a host cell that allows a PRL-3 TRE to function, such as a cell (preferably a mammalian cell, more preferably a human cell, even more preferably a metastatic colon cancer cell). The metastatic colon cancer-specific TRE may comprise one or more regulatory sequences, e.g. enhancers, promoters, transcription factor binding sites and the like, which may be derived from the same or different genes. In one preferred aspect, the PRL-3 TRE comprises a PRL-3 promoter. One preferred PRL-3 TRE is derived from the 0.6 kb sequence upstream of the translational start codon for the PRL-3 gene, described in WO 04/009790, expressly incorporated by reference herein.

In another aspect, the invention provides adenoviral vectors comprising a liver cancer specific TREs derived from the CRG-L2 gene operatively linked to a gene essential for adenovirus replication or a transgene. As used herein, a “liver cancer specific TREs derived from the CRG-L2 gene” or a “CRG-L2 TRE” is a polynucleotide sequence, preferably a DNA sequence, which selectively increases transcription of an operatively linked polynucleotide sequence in a host cell that allows a CRG-L2 to function, such as a cell (preferably a mammalian cell, more preferably a human cell, even more preferably a hepatocellular carcinoma cell). The hepatocellular carcinoma specific TRE may comprise one or more regulatory sequences, e.g. enhancers, promoters, transcription factor binding sites and the like, which may be derived from the same or different genes. In one preferred aspect, the CRG-L2 TRE may be derived from the 0.8 kb sequence upstream of the translational start codon for the CRG-L2 gene, or from a 0.7 kb sequence contained within the 0.8 kb sequence (residues 119-803); or from an EcoRI to NcoI fragment derived from the 0.8 kb sequence, as described in U.S. Provisional Application Ser. No. 60/511,812, expressly incorporated by reference herein.

In another aspect, the invention provides adenoviral vectors comprising an EBV-specific transcriptional regulatory element (TRE) operatively linked to a gene essential for adenovirus replication or a transgene. In one aspect, the EBV specific TRE is derived from a sequence upstream of the translational start codon for the LMP1, LMP2A or LMP2B genes, as further described in U.S. Provisional Application Ser. No. 60/423,203, expressly incorporated by reference herein. The EBV-specific TRE may comprise one or more regulatory sequences, e.g. enhancers, promoters, transcription factor binding sites and the like, which may be derived from the same or different genes.

In yet another aspect, the invention provides adenoviral vectors comprising a hypoxia-responsive element (“HRE”) operatively linked to a gene essential for adenovirus replication or a transgene. HRE is a transcriptional regulatory element comprising a binding site for the transcriptional complex HIF-1, or hypoxia inducible factor-1, which interacts with a in the regulatory regions of several genes, including vascular endothelial growth factor, and several genes encoding glycolytic enzymes, including enolase-1. Accordingly, in one embodiment, an adenovirus vector comprises an adenovirus gene, preferably an adenoviral gene essential for replication, under transcriptional control of a cell status-specific TRE such as a HRE, as further described in WO 00/15820, expressly incorporated by reference herein.

In yet another aspect, the invention provides adenoviral vectors comprising a “telomerase promoter” or “TERT promoter” operatively linked to a gene essential for adenovirus replication or a transgene. The term “telomerase promoter” or “TERT promoter” as used herein refers to a native TERT promoter and functional fragments, mutations and derivatives thereof. The TERT promoter does not have to be the full-length or wild type promoter. One skilled in the art knows how to derive fragments from a TERT promoter and test them for the desired selectivity. A TERT promoter fragment of the present invention has promoter activity selective for tumor cells, i.e. drives tumor selective expression of an operatively linked coding sequence. In one embodiment, the TERT promoter of the invention is a mammalian TERT promoter. In another embodiment, the mammalian TERT promoter is a human TERT (hTERT) promoter. See, e.g., WO 98/14593 and WO 00/46355 for exemplary TERT promoters that find utility in the compositions and methods of the present invention.

In yet another aspect, the invention provides adenoviral vectors comprising an “E2F promoter” operatively linked to a gene essential for adenovirus replication or a transgene. The term “E2F promoter” as used herein refers to a native E2F promoter and functional fragments, mutations and derivatives thereof. The E2F promoter does not have to be the full-length or wild type promoter. One skilled in the art knows how to derive fragments from an E2F promoter and test them for the desired selectivity. An E2F promoter fragment of the present invention has promoter activity selective for tumor cells, i.e. drives tumor selective expression of an operatively linked coding sequence. A number of examples of E2F promoters are known in the art. See, e.g., Parr et al. Nature Medicine 1997:3(10) 1145-1149, WO 02/067861, US20010053352 and WO 98/13508.

The protein urokinase plasminogen activator (uPA) and its cell surface receptor, urokinase plasminogen activator receptor (uPAR), are expressed in many of the most frequently occurring neoplasms and appear to represent important proteins in cancer metastasis. Both proteins are implicated in breast, colon, prostate, liver, renal, lung and ovarian cancer. Sequence elements that regulate uPA and uPAR transcription have been extensively studied. (Riccio et al. (1985) Nucleic Acids Res. 13:2759-2771; Cannio et al. (1991) Nucleic Acids Res. 19:2303-2308; See also, WO 98/39464).

Adenoviral Vector Preparation and Production

Standard systems for generating adenoviral vectors for expression of inserted sequences are known in the art and are available from commercial sources, for example the Adeno-X™ expression system from Clontech (Palo Alto, Calif.) (Clontechniques (January 2000) p. 10-12), the Adenovator™ Adenoviral Vector System and AdEasy™, both from Qbiogene (Carlsbad, Calif.).

For convenience, plasmids are available that provide the necessary portions of adenovirus. Plasmid pXC.1 (McKinnon (1982) Gene 19:33-42) contains the wild-type left-hand end of Ad5. pBHG10 (Bett et al. (1994); Microbix Biosystems Inc., Toronto) provides the right-hand end of Ad5, with a deletion in E3. pBHG11 provides an even larger E3 deletion, an additional 0.3 kb is deleted (Bett et al. (1994). Alternatively, the use of pBHGE3 (Microbix Biosystems, Inc.) provides the right hand end of Ad5, with a full-length of E3.

For manipulation of the early genes, the transcription start site of Ad5 E1A is at 498 and the ATG start site of the E1A coding segment is at 560 in the virus genome. This region can be used for insertion of a heterologous TRE.

A restriction site may be introduced by employing polymerase chain reaction (PCR), where the primer that is employed may be limited to the Ad5 genome, or may involve a portion of the plasmid carrying the Ad5 genomic DNA. For example, where pBR322 is used, the primers may use the EcoRI site in the pBR322 backbone and the XbaI site at nt 1339 of Ad5. By carrying out the PCR in two steps, where overlapping primers at the center of the region introduce a nucleotide sequence change resulting in a unique restriction site, one can provide for insertion of a heterologous TRE at that site.

A similar strategy may also be used for insertion of a heterologous TRE element in operative linkage to E1B. The E1B promoter of Ad5 consists of a single high-affinity recognition site for Sp1 and a TATA box. This region extends from Ad5 nt 1636 to 1701. By insertion of a cell-specific heterologous TRE in this region, one can provide for cell-specific transcription of the E1B gene. By employing the left-hand region modified with the cell-specific response element regulating E1A, as the template for introducing a heterologous TRE to regulate E1B, the resulting adenovirus vector will be dependent upon the cell-specific transcription factors for expression of both E1A and E1B. In some embodiments, part or all of the 19-kDa region of E1B is deleted.

Similarly, a cell-specific heterologous TRE can be inserted upstream of the E2 gene to make its expression cell-specific. The E2 early promoter, mapping in Ad5 from about 27050-27150, consists of a major and a minor transcription initiation site, the latter accounting for about 5% of the E2 transcripts, two non-canonical TATA boxes, two E2F transcription factor binding sites and an ATF transcription factor binding site (for a detailed review of the E2 promoter architecture see Swaminathan et al., Curr. Topics in Micro. and Immunol. (1995) 199(part 3):177-194.

The E2 late promoter overlaps with the coding sequences of a gene encoded by the counterstrand and is therefore not amenable for genetic manipulation. However, the E2 early promoter overlaps only for a few base pairs with sequences coding for a 33 kD protein on the counterstrand. Notably, the SpeI restriction site (Ad5 position 27082) is part of the stop codon for the above mentioned 33 kD protein and conveniently separates the major E2 early transcription initiation site and TATA-binding protein site from the upstream transcription factor binding sites E2F and ATF. Therefore, insertion of a heterologous TRE having SpeI ends into the SpeI site would disrupt the endogenous E2 early promoter of Ad5 and should allow cell-specific expression of E2 transcripts.

For E4, one must use the right hand portion of the adenovirus genome. The E4 transcription start site is predominantly at about nt 35605 for Ad5, the TATA box at about nt 35631 and the first AUG/CUG of ORF I is at about nt 35532. Virtanen et al. (1984) J. Virol. 51: 822-831. Using any of the above strategies for the other genes, a heterologous TRE may be introduced upstream from the transcription start site. For the construction of full-length adenovirus with a heterologous TRE inserted in the E4 region, the co-transfection and homologous recombination may be performed in W162 cells (Weinberg et al. (1983) Proc. Natl. Acad. Sci. 80:5383-5386) which provide E4 proteins in trans to complement defects in synthesis of these proteins.

In one embodiment, the invention provides adenovirus vectors in which an adenovirus gene is under transcriptional control of a first TRE and a polynucleotide sequence encoding an ADP under control of a second TRE element, and wherein preferably the adenovirus gene is essential for replication. The DNA sequence encoding ADP and the amino acid sequence of an ADP are publicly available. Briefly, an ADP coding sequence is obtained from Ad using techniques known in the art, such as PCR. Preferably, the Y leader (which is an important sequence for correct expression of late genes) is also obtained and ligated to the ADP coding sequence. The ADP coding sequence (with or without the Y leader) can then be introduced into the adenoviral genome, for example, in the E3 region (where the ADP coding sequence will be driven by the MLP). The ADP coding sequence could also be inserted in other locations of the adenovirus genome, such as the E4 region. Alternatively, the ADP coding sequence could be operatively linked to a different type of TRE, including, but not limited to, another viral TRE. In one embodiment, the vector of the invention has ADP operatively linked to its native TREs.

The viral vectors of this invention can be prepared using recombinant techniques that are standard in the art. Methods of modifying replication-competent or replication-incompetent viral vectors are well known in the art and are described herein and in publications cited herein. Various methods for modifying adenoviral vectors and cloning transgenes and desired transcriptional elements into adenovirus are described herein and are standard and well know in the art. There are various plasmids in the art that contain the different portions of the adenovirus genome, including plasmids that contain the entire adenovirus genome. The construction of these plasmids is also well described in the art (e.g. US20030104625). Once a site is selected for modification an appropriate plasmid can be used to perform the modifications. Then the modifications may be introduced into a full-length adenoviral vector genome by, for example homologous recombination or in vitro ligation. The homologous recombination, for example, may take place in a mammalian cell (e.g. PerC6) or in a bacterial cell (e.g. E. Coli, see WO9617070). Manipulation of the viral vector genome can alternatively or in addition include well known molecular biology methods including, but not limited to, polymerase chain reaction (PCR), PCR-SOEing and restriction digests. If homologous recombination is employed, the two plasmids should share at least about 500 bp of sequence overlap, although smaller regions of overlap will recombine, but usually with lower efficiencies. Each plasmid, as desired, may be independently manipulated, followed by cotransfection in a competent host, providing complementing genes as appropriate for propagation of the adenoviral vector. Plasmids are generally introduced into a suitable host cell (e.g. 293, PerC.6, Hela-S3 cells) using appropriate means of transduction, such as cationic liposomes or calcium phosphate. Alternatively, in vitro ligation of the right and left-hand portions of the adenovirus genome can also be used to construct recombinant adenovirus derivative containing all the replication-essential portions of adenovirus genome. Berkner et al. (1983) Nucleic Acid Research 11: 6003-6020; Bridge et al. (1989) J. Virol. 63: 631-638.

“Producer cells” for viral vectors are well known in the art (e.g. PCT/US98/04080). A producer cell is a cell in which the adenoviral vector is delivered and the adenoviral vector is replicated and packaged into virions. The preferred packaging cells are those that have been designed to limit homologous recombination that could lead to wildtype adenoviral particles. If the viral vector has an essential gene deleted or inactivated, then the producer cell complements for the inactivated gene. Cells that may be used to produce the adenoviral particles of the invention include the human embryonic kidney cell line 293 (Graham et al., J Gen. Virol. 36:59-72 (1977)), the human embryonic retinoblast cell line PER.C6 (U.S. Pat. Nos. 5,994,128 and 6,033,908; Fallaux et al., Hum. Gene Ther. 9: 1909-1917 (1998)), and the human cervical tumor-derived cell line HeLa-S3 (PCT Application NO. US 04/11855). Alternatively or in addition, the producer cell may express the genes that are selectively controlled or inactivated in the viral vector. One embodiment of the invention includes a producer cell which contains an adenoviral vector of the present invention.

Chimeric Adenoviral Fiber Proteins

The adenovirus fiber protein plays an important role for attachment of the virion to cellular receptors. The sequences of the fiber genes from several different adenovirus serotypes are known in the art. The fiber protein is divided into three domains. The conserved N terminus contains the sequences responsible for association with the penton base as well as a nuclear localization signal. A rod-like “shaft” of variable length contains repeats of a 15-amino-acid beta structure, with the number of repeats ranging from 6 in Ad3 to 22 in Ad5. A conserved stretch of amino acids which includes the sequence TLWT marks the boundary between the repeating units of beta structure in the shaft and the globular head domain. The C-terminal head domain ranges in size from 157 amino acid residues for the short fiber of Ad41 to 188 residues in the Ad5 fiber. The fiber spike is a homotrimer, and there are 12 spikes per virion which are attached via association with the penton base complex. The fiber protein must be able to trimerize in order to be functional. The trimerization domain may be the native domain or a heterologous trimerization domain.

The chimeric adenoviral fibers that are useful in the present invention comprise at least a portion of Subgroup C adenovirus (e.g. Ad1, 2, 5 and 6 serotype) shaft region and at least a portion of the head region of a Subgroup B adenovirus (e.g. Ad3, 7, 11, 14, 16, 21, 35 and 50) wherein the head region binds CD46. In one embodiment, the complete shaft region is from Ad2 or Ad5 and the complete head region is from Ad35. In another embodiment, the chimeric fiber comprises a portion of a Subgroup C adenovirus shaft region and a head region from an adenovirus that binds CD46. Fiber head regions that bind CD46 include, but are not limited to, those derived from Ad3 (Sirena et al., J Virol. 2004 May; 78(9):4454-62), 11, 14, 16, 21, 35, 37 and 50 In another embodiment, the chimeric fiber protein comprises a bovine adenovirus shaft domain and a portion of a head domain derived from an adenovirus serotype selected from the group consisting of Ad3, 7, 11, 14, 16, 21, 35, 37, 50 and a Subgroup D adenovirus. In another embodiment, the chimeric fiber protein comprises a portion of a Subgroup C adenovirus shaft region and a portion of a Subgroup D adenovirus head region. In the examples below, it is demonstrated that an Ad2 or Ad5 derived adenoviral vector with a chimeric fiber comprising an Ad2 or Ad5 shaft and an Ad35 head region transduces certain primary tumor cells more efficiently than the same adenoviral vector with either a native Ad5 or Ad35 fiber protein. One skilled in the art will realize that further modifications may be made to the chimeric fiber protein while retaining the enhanced transduction of primary tumor cells.

GenBank AAA75331 discloses the sequence of an Ad35 fiber. This is an exemplary sequence and a number of genomic variants exist (Flomenberg et al., J. Infec. Dis., 155(6) 1127-1134 (1987)). In practicing the present invention, the portion of the adenoviral protein derived from Ad35 head region may be from any Ad35 genomic variant.

Given the Ad2, Ad5 and Ad35 sequence information known in the art and the references instruction provided herein, one skilled in the art can combine the appropriate portions of the Ad2 or Ad5 shaft and portions of an Ad35 head in order to achieve enhanced transduction of tumor cells, in particular primary tumor cells. For example, one skilled in the art can perform deletion and substitution analysis to determine how to combine the various sequence portions.

In one embodiment, the chimeric fiber protein comprises the complete adenovirus serotype 5 (Ad5) fiber shaft (amino acids 47 to 399 of SEQ ID NO:2) or the complete Ad2 fiber shaft (amino acids 47 to 399 of SEQ ID NO:4). In another embodiment, the chimeric fiber protein comprises the head region from an adenovirus serotype 35 fiber protein (amino acids 46 to 132 of SEQ ID NO:6). In other embodiments, the chimeric fiber protein comprises the complete adenovirus serotype 5 (Ad5) fiber shaft (amino acids 47 to 399 of SEQ ID NO:2) or the complete Ad2 fiber shaft (amino acids 47 to 399 of SEQ ID NO:4) and the head region from an adenovirus serotype 35 fiber protein (amino acids 46 to 132 of SEQ ID NO:6). In another embodiment, the chimeric fiber protein comprises a sequence portion derived from an Ad2 or Ad5 fiber shaft and a sequence portion derived from the head region of Ad35.

In one embodiment, the Ad5 or Ad2 shaft region retains the KKTK sequence (amino acids 91-94 of SEQ ID NO:2 or SEQ ID NO:4). In an alternative embodiment the KKTK sequence in the native shaft sequence is deleted or mutated. In one embodiment, the Ad5 shaft retains the KLGTGLSFD sequence (amino acid 376-384 of SEQ ID NO:2) (Wu et al. J Virol. July 2003; 77(13):7225-35), the Ad2 shaft retains the KLGAGLSFD sequence (amino acids 376-384 of SEQ ID NO:4) or the shaft contains the consensus motif KLGXGLXFD/N (SEQ ID NO:7; Wu et al. 2003). In one embodiment, the Ad5 shaft retains the GNLTSQNVTTVSPPLKKTK comprising the third repeat region of the shaft with flexibility domain (amino acids 76-94 of SEQ ID NO: 2). In an alternative embodiment, Ad35 shaft contains the third repeat of the shaft (GTLQENIRATAPITKNN), which lacks the sequence responsible for flexibility of the fiber (amino acids 76-92 of SEQ ID NO: 6)

The chimeric fiber proteins may include further modifications including, but not limited to modifications that decrease binding of the viral vector particle to a particular cell type or more than one cell type, enhance the binding of the viral vector particle to a particular cell type or more than one cell type and/or reduce the immune response to the adenoviral vector in an animal. Examples of these modifications include, but are not limited to those described in U.S. application Ser. No. 10/403,337, WO 98/07877, WO 01/92299, WO 2003/62400 and U.S. Pat. Nos. 5,962,311, 6,153,435, 6,455,314 and Wu et al. (J Virol. Jul. 1, 2003;77(13):7225-7235).

A non-native ligand may be included in the HI loop or at the carboxyl end of the chimeric fiber protein

Self-Processing Cleavage Sites and 2A-Like Sequences

In another aspect of the invention a “self-processing cleavage site” (e.g. 2A-like sequence) is utilized to express two polypeptides from one mRNA. A “self-processing cleavage site” or “self-processing cleavage sequence” is defined as a DNA or amino acid sequence, wherein upon translation, rapid intramolecular (cis) cleavage of a polypeptide comprising the self-processing cleavage site occurs to result in expression of discrete mature protein or polypeptide products. Such a “self-processing cleavage site”, may also be referred to as a post-translational or co-translational processing cleavage site, exemplified herein by a 2A site, sequence or domain. As used herein, a “self-processing peptide” is defined herein as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products. It has been reported that a 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly et al. J Gen Virol. 2001 May; 82(Pt 5):1013-25). Alternatively, it has also been reported that a 2A site, sequence or domain demonstrates “auto-proteolysis” or “cleavage” by cleaving its own C-terminus in cis to produce primary cleavage products (Furler; Palmenberg, Ann. Rev. Microbiol. 44:603-623 (1990)).

Variations of the 2A sequence have been studied for their ability to mediate efficient processing of polyproteins (Donnelly et al., J. Gen. Virol. 82:1027-1041 (2001)). Homologues and variant 2A sequences are included within the scope of the invention.

Vector constructs that comprise a sequence encoding a self-processing cleavage sequence, such as a 2A or 2A-like sequence between open reading frames may further comprise an additional proteolytic cleavage site adjacent to the self-processing cleavage sequence for removal of amino acids that comprise the self-processing cleavage sequence following cleavage. The term “additional proteolytic cleavage site”, refers to a sequence which is incorporated into an adenoviral vector of the invention adjacent a self-processing cleavage site, such as a 2A or 2A like sequence, and provides a means to remove additional amino acids that remain following cleavage by the self processing cleavage sequence. Exemplary “additional proteolytic cleavage sites” are described herein and include, but are not limited to, furin cleavage sites with the consensus sequence RXK(R)R. Vector constructs that comprise a sequence encoding a self-processing cleavage sequence, such as a 2A or 2A-like sequence between open reading frames and may further comprise an additional proteolytic cleavage site, are further described in U.S. patent application Ser. No. 10/831,302, expressly incorporated by reference herein.

In one embodiment, the invention provides a method for removal of residual amino acids and a composition for expression of the same. A number of novel constructs have been designed that provide for removal of additional amino acids from the C-terminus of the protein. Furin cleavage occurs at the C-terminus of the cleavage site, which has the consensus sequence RXR(K)R, where X is any amino acid. In one aspect, the invention provides a means for removal of the newly exposed basic amino acid residues R or K from the C-terminus of the protein by use of an enzyme selected from a group of enzymes called carboxypeptidases (CPs), which include, but not limited to, carboxypeptidase D, E and H (CPD, CPE, CPH). Since CPs are able to remove basic amino acid residues at the C-terminus of a protein, all amino acid resides derived from a furin cleavage site which contain exclusively basic amino acids R or K, such as RKKR, RKRR, RRRR, etc, can be removed by a CP.

In one embodiment of the invention, a self-processing cleavage sequence (e.g. 2A or 2A-like sequence) is operatively linked to an adenovirus protein coding region and a transgene. The adenovirus protein CDS may be upstream of the self-processing cleavage site, with the transgene being downstream. Alternatively, the transgene CDS may be upstream of the self-processing cleavage site, with the adenovirus protein CDS being downstream.

Multiple CDSs may be linked with self-processing cleavage sites. In one embodiment, an Ad CDS is operatively linked by a self-processing cleavage site to a first transgene and said first transgene is operatively linked by a self-processing cleavage site to a second transgene. In one embodiment, the first and second transgenes encodes for the same or different proteins

Transgenes

The vectors of the invention may include one or more transgenes. In this way, various genetic capabilities may be introduced into target cells.

A “cytokine” or grammatical equivalent, includes, without limitation, those hormones that act locally and do not circulate in the blood, and which, when used in accordance with the present invention, will result in an alteration of an individual's immune response. Also included in the definition of cytokine are adhesion or accessory molecules which result in an alteration of an individual's immune response. Thus, examples of cytokines include, but are not limited to, IL-1(a or P), IL-2, IL-3, IL-4, IL-5, IL-6, IL-7, IL-8, IL-9, IL-10, IL-11, IL-12, GM-CSF, M-CSF, G-CSF, LIF, LT, TGF-P, y-IFN, a-IFN, P-IFN, TNF-a, BCGF, CD2, or ICAM. Descriptions of the aforementioned cytokines as well as other applicable immunomodulatory agents may be found in “Cytokines and Cytokine Receptors,” A. S. Hamblin, D. Male (ed.), Oxford University Press, New York, N.Y. (1993)), or the “Guidebook to Cytokines and Their Receptors,” N. A. Nicola (ed.), Oxford University Press, New York, N.Y. (1995)). Where therapeutic use in humans is contemplated, the cytokines will preferably be substantially similar to the human form of the protein or will have been derived from human sequences (i.e., of human origin). In one preferred embodiment, the transgene is a cytokine, such as GM-CSF.

Additionally, cytokines of other mammals with substantial structural homology and/or amino acid sequence identity to the human forms of IL-2, GM-CSF, TNF-a, and others, will be useful in the invention when demonstrated to exhibit similar activity on the immune system. Similarly, proteins that are substantially analogous to any particular cytokine, but have conservative changes of protein sequence, will also find use in the present invention. Thus, conservative substitutions in protein sequence may be possible without disturbing the functional abilities of the protein molecule, and thus proteins can be made that function as cytokines in the present invention but have amino acid sequences that differ slightly from currently known sequences. Such conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

Finally, the use of either the singular or plural form of the word “cytokine” in this application is not determinative and should not limit interpretation of the present invention and claims. In addition to the cytokines, adhesion or accessory molecules or combinations thereof, may be employed alone or in combination with the cytokines.

Granulocyte-macrophage colony stimulating factor (GM-CSF) is a cytokine produced by fibroblasts, endothelial cells, T cells and macrophages. This cytokine has been shown to induce the growth of hematopoetic cells of granulocyte and macrophage lineages. In addition, it also activates the antigen processing and presenting function of dendritic cells, which are the major antigen presenting cells (APC) of the immune system. Results from animal model experiments have convincingly shown that GM-CSF producing tumor cells (i.e. GVAX) are able to induce an immune response against parental, non-transduced tumor cells.

GM-CSF augments the antigen presentation capability of the subclass of dendritic cells (DC) capable of stimulating robust anti-tumor responses (Gasson et al. Blood Mar. 15, 1991;77(6):1131-45; Mach et al. Cancer Res. Jun. 15, 2000;60(12):3239-46; reviewed in Mach and Dranoff, Curr Opin Immunol. October 2000;12(5):571-5). See, e.g., Boon and Old, Curr Opin Immunol. Oct. 1, 1997; 9(5):681-3). Presentation of tumor antigen epitopes to T cells in the draining lymph nodes is expected to result in systemic immune responses to tumor metastases. Also, irradiated tumor cells expressing GM-CSF have been shown to function as potent vaccines against tumor challenge (as further described in the section below, entitled “GVAX”). Localized high concentrations of certain cytokines, delivered by genetically modified cells, have been found to lead to tumor regression (Abe et al., J. Canc. Res. Clin. Oncol. 121: 587-592 (1995); Gansbacher et al., Cancer Res. 50: 7820-7825 (1990); Forni et al., Cancer and Met. Reviews 7: 289-309 (1988). PCT publication WO200072686 describes tumor cells expressing various cytokines.

In one embodiment of the invention, the adenovirus comprises a GM-CSF coding sequence operatively linked to regulatory elements for expression in the primary tumor cell. In one embodiment, the GM-CSF coding sequence codes for a human GM-CSF. Alternatively, the GM-CSF coding sequence may code for a murine GM-CSF. In some embodiments, the coding sequence for GM-CSF is a cDNA sequence (e.g. SEQ ID NO:16). In other words, the coding sequence for GM-CSF does not contain intronic sequences to be spliced out prior to translation. In another embodiment, the coding sequence for GM-CSF is a genomic coding sequence (e.g. SEQ ID NO:15). In other words, the coding sequence contains at least one native GM-CSF intron that is spliced out prior to translation. In one embodiment, the GM-CSF coding sequence codes for SEQ ID NO:14. Other examples of GM-CSF coding sequences are found in Genbank accession numbers: AF373868, AC034228, AC034216, M10663 and NM000758.

In one embodiment, the transgene encodes a marker. In another embodiment, the transgene encodes a cytotoxic protein. Vectors encoding a cytotoxic protein may be used to enhance the degree of therapeutic efficacy by enhancing the rate of cytotoxic activity. This can be accomplished by coupling regulated viral replication and corresponding selective cytotoxicity with expression of, one or more metabolic enzymes such as HSV-tk, nitroreductase, cytochrome P450 or cytosine deaminase (CD) which render cells capable of metabolizing 5-fluorocytosine (5-FC) to the chemotherapeutic agent 5-fluorouracil (5-FU), carboxylesterase (CA), deoxycytidine kinase (dCK), purine nucleoside phosphorylase (PNP), carboxypeptidase G2 (CPG2; Niculescu-Duvaz et al. J Med Chem. May 6, 2004;47(10):2651-2658), thymidine phosphorylase (TP), thymidine kinase (TK) or xanthine-guanine phosphoribosyl transferase (XGPRT). This type of transgene may also be used to confer a bystander effect.

Additional examples of transgenes that may be included in an adenoviral vector of the invention include a factor capable of initiating apoptosis, antisense or ribozymes, which among other capabilities may be directed to mRNAs encoding proteins essential for proliferation of the cells or a pathogen, such as structural proteins, transcription factors, polymerases, etc., viral or other pathogenic proteins, where the pathogen proliferates intracellularly, cytotoxic proteins, e.g., the chains of diphtheria, ricin, abrin, etc., genes that encode an engineered cytoplasmic variant of a nuclease (e.g., RNase A) or protease (e.g., trypsin, papain, proteinase K, carboxypeptidase, etc.), chemokines, such as MCP3 alpha or MIP-1, pore-forming proteins derived from viruses, bacteria, or mammalian cells, fusgenic genes, chemotherapy sensitizing genes and radiation sensitizing genes. Other genes of interest include cytokines, antigens, transmembrane proteins, and the like, such as IL-1, IL-2, IL-4, IL-5, IL-6, IL-10, IL-12, IL-18 or flt3, GM-CSF, G-CSF, M-CSF, IFN-α, -β, -γ, TNF-α, -β, TGF-α, -β, NGF, MDA-7 (Melanoma differentiation associated gene-7, mda-7/interleukin-24), and the like. Further examples include, proapoptotic genes such as Fas, Bax, Caspase, TRAIL, Fas ligands, nitric oxide synthase (NOS) and the like; fusion genes which can lead to cell fusion or facilitate cell fusion such as V22, VSV and the like; tumor suppressor gene such as p53, RB, p16, p17, W9 and the like; genes associated with the cell cycle and genes which encode anti-angiogenic proteins such as endostatin, angiostatin and the like.

Although any gene or coding sequence of relevance can be used in the practice of the invention, certain genes, or fragments thereof, are particularly suitable. For example, coding regions encoding immunogenic polypeptides, toxins, immunotoxins and cytokines are useful in the practice of the invention. These coding regions include those hereinabove and additional coding regions include those that encode the following: proteins that stimulate interactions with immune cells such as B7, CD28, MHC class I, MHC class II, TAPs, tumor-associated antigens such as immunogenic sequences from MART-1, gp 100 (pmel-17), tyrosinase, tyrosinase-related protein 1, tyrosinase-related protein 2, melanocyte-stimulating hormone receptor, MAGE1, MAGE2, MAGE3, MAGE12, BAGE, GAGE, NY-ESO-1, β-catenin, MUM-1, CDK-4, caspase 8, KIA 0205, HLA-A2R1701, α-fetoprotein, telomerase catalytic protein, G-250, MUC-1, carcinoembryonic protein, p53, Her2/neu, triosephosphate isomerase, CDC-27, LDLR-FUT, telomerase reverse transcriptase, PSMA, cDNAs of antibodies that block inhibitory signals (CTLA4 blockade), chemokines (MIP1α, MIP3α, CCR7 ligand, and calreticulin), anti-angiogenic genes include, but are not limited to, genes that encode METH-1, METH -2, TrpRS fragments, proliferin-related protein, prolactin fragment, PEDF, vasostatin, various fragments of extracellular matrix proteins and growth factor/cytokine inhibitors, various fragments of extracellular matrix proteins which include, but are not limited to, angiostatin, endostatin, kininostatin, fibrinogen-E fragment, thrombospondin, tumstatin, canstatin, restin, growth factor/cytokine inhibitors which include, but are not limited to, VEGFJVEGFR antagonist, sFlt-1, sFlk, sNRP1, angiopoietin/tie antagonist, sTie-2, chemokines (IP-10, PF-4, Gro-beta, IFN-gamma (Mig), IFNα, FGF/FGFR antagonist (sFGFR), Ephrin/Eph antagonist (sEphB4 and sephrinB2), PDGF, TGFβ and IGF-1. Genes suitable for use in the practice of the invention can encode enzymes (such as, for example, urease, renin, thrombin, metalloproteases, nitric oxide synthase, superoxide dismutase, catalase and others known to those of skill in the art), enzyme inhibitors (such as, for example, alphal-antitrypsin, antithrombin III, cellular or viral protease inhibitors, plasminogen activator inhibitor-1, tissue inhibitor of metalloproteases, etc.), the cystic fibrosis transmembrane conductance regulator (CFTR) protein, insulin, dystrophin, or a Major Histocompatibility Complex (MHC) antigen of class I or II. Also useful are genes encoding polypeptides that can modulate/regulate expression of corresponding genes, polypeptides capable of inhibiting a bacterial, parasitic or viral infection or its development (for example, antigenic polypeptides, antigenic epitopes, and transdominant protein variants inhibiting the action of a native protein by competition), apoptosis inducers or inhibitors (for example, Bax, Bc12, Bc1X and others known to those of skill in the art), cytostatic agents (e.g., p21, p16, Rb, etc.), apolipoproteins (e.g., ApoAI, ApoAIV, ApoE, etc.), oxygen radical scavengers, polypeptides having an anti-tumor effect, antibodies, toxins, immunotoxins, markers (e.g., beta-galactosidase, luciferase, etc.) or any other genes of interest that are recognized in the art as being useful for treatment or prevention of a clinical condition. Further transgenes include those coding for a polypeptide which inhibits cellular division or signal transduction, a tumor suppressor protein (such as, for example, p53, Rb, p73), a polypeptide which activates the host immune system, a tumor-associated antigen (e.g., MUC-1, BRCA-1, an HPV early or late antigen such as E6, E7, L1, L2, etc), optionally in combination with a cytokine.

The invention further includes adenoviral vectors comprising combinations of two or more transgenes. In some cases the two or more transgenes have synergistic, complementary and/or nonoverlapping toxicities and methods of action.

In designing the adenoviral vectors of the invention the biological activity of the transgene is considered, e.g. in some cases it is advantageous that the transgene be inserted in the vector such that the transgene is only expresses or is mostly expressed at the early or late stages of adenoviral infection. For some transgenes, it may be preferred to express the transgene early in the viral life cycle. In such cases, the transgene may be inserted in any of the early regions (for example, E3) or into the upstream L1 region.

GVAX

The present invention also relates to a method of altering an individual's immune response to a target antigen or antigens by providing tumor cells transduced with an adenoviral vector that has a chimeric fiber protein wherein the chimeric fiber protein comprises at least a portion of an Ad5 or Ad2 shaft and at least a portion of an Ad35 head. Typically the tumor cell is a primary tumor cell. Exemplary tumor cells include tumor cells from the same individual (autologous) or from a different individual (allogeneic). In another embodiment, the tumor cell is from a tumor cell line of the same type as the tumor or cancer being treated.

The invention further includes a method of increasing the immune response to a tumor cell by administering to a mammal, tumor cells transduced with an adenoviral vector that has a chimeric fiber protein wherein the chimeric fiber protein comprises at least a portion of an Ad5 or Ad2 shaft and at least a portion of an Ad35 head, wherein the mammal's immune response to a tumor cell is increased. In one embodiment, the increased immune response is humoral. In another embodiment, the increased immune response is cellular. In yet another embodiment, the increased immune response is both cellular and humoral.

In a related embodiment, the method further comprises inactivating the transduced tumor cells and administering the to a mammal. The administered tumor cells may be an autologous transduced tumor cells, allogeneic or bystander cells (as further defined below). Typically the transduced tumor cells are rendered proliferation incompetent prior to administration. In one embodiment, the mammal is a human who harbors tumor cells of the same type as the transduced tumor cells. In a preferred embodiment, the tumor cells of the mammal exhibit growth inhibition or cell death subsequent to administration of the transduced tumor cells.

In still another aspect, the invention provides a method for stimulating a systemic immune response in a mammal to a tumor, or to an antigen thereof, by administering a therapeutically effective amount of proliferation incompetent tumor cells that have been genetically modified by transduction with a chimeric recombinant adenovirus comprising a nucleic acid encoding at least one human cytokine, wherein the transduced tumor cells and the tumor are of the same type and express at least one common antigen.

In certain embodiments, the systemic immune response to the tumor results in tumor regression or inhibits the growth of the tumor.

In one embodiment, the tumor cell is derived from a mammal, such as a human, harboring a tumor. In some embodiments, the tumor cells are cryopreserved prior to administration. In certain embodiments, the type of tumor cell being treated is selected from the group consisting of prostate tumor, melanoma, non-small cell lung carcinoma, breast cancer, epidermal cancer, bladder cancer, prostate cancer, head and neck cancer, pre-neoplastic lesion, cancerous polyp, ovarian cancer, cervical cancer, leukemia, and renal carcinoma. When the type of tumor cell being treated is prostate cancer, the prostate tumor cell line may be selected from the group consisting of DU145, PC-3, and LnCaP.

In one preferred embodiment of the invention, the adenoviral vector of the invention is utilized to deliver a human GM-CSF transgene to a primary human tumor cell ex vivo. After transduction, the cells are irradiated to render them proliferation incompetent. The proliferation incompetent GM-CSF expressing cells are then re-administered to the patient (e.g., by the intradermal or subcutaneous route) and thereby function as a cancer vaccine.

The tumor cell is selected from the group consisting of an autologous tumor cell, an allogeneic tumor cell and a tumor cell line. The tumor cells may be transduced in vitro, ex vivo or in vivo. Autologous and allogeneic cancer cells that have been genetically modified to express a cytokine, e.g., GM-CSF, followed by readministration to a patient for the treatment of cancer are described in U.S. Pat. Nos. 5,637,483, 5,904,920 and 6,350,445, expressly incorporated by reference herein. A form of GM-CSF-expressing genetically modified tumor cells or a “cytokine-expressing cellular vaccine” (“GVAX”), for the treatment of pancreatic cancer is described in U.S. Pat. Nos. 6,033,674 and 5,985,290, expressly incorporated by reference herein. A universal immunomodulatory genetically modified bystander cell line is described in U.S. Pat. No. 6,464,973, expressly incorporated by reference herein.

Clinical trials employing GM-CSF-expressing cellular vaccines (GVAX) have been undertaken for treatment of prostate cancer, melanoma, lung cancer, pancreatic cancer, renal cancer, and multiple myeloma. A number of clinical trials using GVAX cellular vaccines have been described, most notably in melanoma, and prostate, renal and pancreatic carcinoma (Simons J W et al. Cancer Res. 1999; 59:5160-5168; Simons J W et al. Cancer Res 1997; 57:1537-1546; Soiffer R et al. Proc. Natl. Acad. Sci USA 1998; 95:13141-13146; Jaffee, et al. J Clin Oncol 2001; 19:145-156; Salgia et al. J Clin Oncol 2003 21:624-30; Soiffer et al. J Clin Oncol 2003 21:3343-50; Nemunaitis et al. J Natl Cancer Inst. Feb. 18, 2004 18 96(4):326-31).

The present invention provides a method of stimulating an improved immune response to cancer in a mammalian subject, preferably a human patient. Desirably, the method effects a systemic immune response, i.e., a T-cell response and/or a B-cell response, to the cancer. The improvement comprises administering to the patient genetically modified tumor cells wherein the tumor cells express at least one antigen expressed by tumor cells harbored by the patient and the genetic modification is accomplished using an adenoviral vector having a chimeric fiber protein wherein the chimeric fiber protein comprises at least a portion of a Subgroup C adenovirus (e.g. Ad1, 2, 5 and 6 serotype) shaft region and at least a portion of the head region of a Subgroup B adenovirus (e.g. Ad3, 7, 11, 14, 16, 21, 35 and 50) wherein the head region binds CD46, e.g., an Ad5 or Ad2 shaft and at least a portion of an Ad35 head. The use of such an adenovirus vector facilitates transduction and transgene expression. The genetically modified tumor cells are rendered proliferation incompetent, typically by irradiation, although any known or later discovered method of rendering the cells proliferation incompetent may be employed. Upon administration of the genetically modified tumor cells to a subject, an immune response to the cancer antigen(s) is stimulated.

In one approach, the genetically modified tumor cells comprise a single population of cells that is modified to express a cytokine and is administered to a subject as part of a treatment regimen. In another approach, two or more populations of genetically modified tumor cells are individually modified to express a different transgene and administered to a subject. The treatment regime may include one or more additional cancer therapeutic agents or treatments.

In general, the genetically modified tumor cells for use in practicing the invention include one or more of autologous tumor cells, allogeneic tumor cells and tumor cell lines (i.e., bystander cells). By way of example, in one approach, the genetically modified tumor cells (i.e., GVAX) is provided as an allogeneic or bystander cell line and one or more additional cancer therapeutic agents is expressed by autologous cells. In another approach, one or more additional transgenes are expressed by an allogeneic or bystander cell line while a cytokine (i.e., GM-CSF) is expressed by autologous cells. Direct comparison of murine tumor cells transduced with various cytokines demonstrated that GM-CSF-secreting tumor cells induced the best overall anti-tumor protection. In one preferred embodiment, the cytokine expressed by the genetically modified tumor cells of the invention is GM-CSF (“GVAX”). The GM-CSF coding sequence is introduced using an improved adenoviral vector which has a chimeric fiber protein comprising at least a portion of a Subgroup C adenovirus shaft region and at least a portion of the head region of a Subgroup B adenovirus wherein the head region binds CD46, e.g., an Ad5 or Ad2 shaft and at least a portion of an Ad35 head. The preferred coding sequence for GM-CSF is the genomic sequence described in Huebner K. et al., Science 230(4731):1282-5,1985, however, in some cases the cDNA form of GM-CSF finds utility in practicing the invention (Cantrell et al., Proc. Natl. Acad. Sci., 82, 6250-6254, 1985).

In some embodiments, the genetically modified tumor cells are cryopreserved prior to administration. In a preferred embodiment, the genetically modified tumor cells are administered to the same individual from whom they were derived. In another preferred embodiment, the genetically modified tumor cells and the tumor are derived from different individuals. In certain preferred embodiments, the type of tumor being treated is selected from the group consisting of cancer of the bladder, breast, colon, kidney, liver, lung, ovary, cervix, pancreas, rectum, prostate, stomach, epidermis; a hematopoietic tumor of lymphoid or myeloid lineage; a tumor of mesenchymal origin such as a fibrosarcoma or rhabdomyosarcoma; other tumor types such as melanoma, teratocarcinoma, neuroblastoma, glioma, adenocarcinoma and non-small lung cell carcinoma.

Preferably, the genetically modified tumor cells are irradiated at a dose of from about 50 to about 200 rads/min, even more preferably, from about 120 to about 140 rads/min prior to administration to the patient. Preferably, the cells are irradiated with a total dose sufficient to inhibit substantially 100% of the cells, from further proliferation. Thus, desirably the cells are irradiated with a total dose of from about 10,000 to 20,000 rads, optimally, with about 15,000 rads.

Autologous

The use of autologous genetically modified transgene-expressing tumor cells provides advantages since each patient's tumor expresses a unique set of tumor antigens that can differ from those found on histologically-similar, MHC-matched tumor cells from another patient. See, e.g., Kawakami et al., J. Immunol., 148, 638-643 (1992); Darrow et al., J. Immunol., 142, 3329-3335 (1989); and Hom et al., J. Immunother., 10, 153-164 (1991). In contrast, MHC-matched tumor cells provide the advantage that the patient need not be taken to surgery to obtain a sample of their tumor for genetically modified tumor cell production.

In one preferred aspect, the present invention comprises a method of treating cancer by carrying out the steps of: (a) obtaining tumor cells from a mammalian subject harboring a tumor; (b) modifying the tumor cells to render them capable of producing an increased level of one or more transgenes relative to unmodified tumor cells using an adenoviral vector having a chimeric fiber protein wherein the chimeric fiber protein comprises at least a portion of a Subgroup C adenovirus shaft region and at least a portion of the head region of a Subgroup B adenovirus adenovirus, wherein the head region binds CD46, e.g., an Ad5 or Ad2 shaft and at least a portion of an Ad35 head; (c) rendering the modified tumor cells proliferation incompetent; and (d) readministering the modified tumor cells to the mammalian subject from which the tumor cells were obtained or to a mammal with the same MHC type as the mammal from which the tumor cells were obtained. The administered tumor cells are autologous and MHC-matched to the host. Preferably, the composition is administered subcutaneously, intradermally or intratumorally to the mammalian subject.

A single autologous tumor cell may express more than one transgene or each transgene may be expressed by a different autologous tumor cell. In one aspect of the invention, an autologous tumor cell is modified by introduction of an adenovirus vector comprising a chimeric fiber protein as described herein and a nucleic acid sequence encoding a transgene, e.g., a cytokine, such as GM-CSF, operatively linked to a promoter and expression/control sequences necessary for expression thereof. In another aspect, the same autologous tumor cell or a second autologous tumor cell is modified by introduction of a vector comprising a nucleic acid sequence encoding at least one additional transgene operatively linked to a promoter and expression/control sequences necessary for expression thereof. The nucleic acid sequence encoding the one or more transgenes are introduced into the same or a different autologous tumor cell using the same or a different vector. The nucleic acid sequence encoding the transgene(s) may or may not further comprise a selectable marker sequence operatively linked to a promoter.

Allogeneic

Researchers have sought alternatives to autologous and MHC-matched cells as tumor vaccines, as reviewed by Jaffee et al., Seminars in Oncology, 22, 81-91 (1995). Early tumor vaccine strategies were based on the understanding that the vaccinating tumor cells function as the antigen presenting cells (APCs) that present tumor antigens on their MHC class I and II molecules, and directly activate the T cell arm of the immune system. The results of Huang et al. (Science, 264, 961-965, 1994), indicate that professional APCs of the host rather than the vaccinating tumor cells prime the T cell arm of the immune system by secreting cytokine(s) such as GM-CSF such that bone marrow-derived APCs are recruited to the region of the tumor. The bone marrow-derived APCs take up the whole cellular protein of the tumor for processing, and then present the antigenic peptide(s) on their MHC class I and II molecules, thereby priming both the CD4+ and the CD8+ T cell arms of the immune system, resulting in a systemic tumor-specific anti-tumor immune response. These results suggest that it may not be necessary or optimal to use autologous or MHC-matched tumor cells in order to elicit an anti-cancer immune response and that the transfer of allogeneic MHC genes (from a genetically dissimilar individual of the same species) can enhance tumor immunogenicity. More specifically, in certain cases, the rejection of tumors expressing allogeneic MHC class I molecules resulted in enhanced systemic immune responses against subsequent challenge with the unmodified parental tumor, as reviewed in Jaffee et al., supra, and Huang et al., supra.

As described herein, a “tumor cell line” comprises cells that were initially derived from a tumor. Such cells typically are transformed (i.e., exhibit indefinite growth in culture). In one preferred aspect, the invention provides a method for treating cancer by carrying out the steps of: (a) obtaining a tumor cell line; (b) modifying the tumor cell line to render the cells capable of producing an increased level of one or more transgenes relative to unmodified tumor cells using an adenoviral vector having a chimeric fiber protein wherein the chimeric fiber protein comprises at least a portion of a Subgroup C adenovirus shaft region and at least a portion of the head region of a Subgroup B adenovirus adenovirus, wherein the head region binds CD46, e.g., an Ad5 or Ad2 shaft and at least a portion of an Ad35 head; (c) rendering the modified tumor cell line proliferation incompetent; and (d) administering the tumor cell line to a mammalian subject (host) having at least one tumor that is of the same type of tumor as that from which the tumor cell line was obtained. The administered tumor cell line is allogeneic and is not MHC-matched to the host. Such allogeneic lines provide the advantage that they can be prepared in advance, characterized, aliquoted in vials containing known numbers of transgene-expressing cells and stored (i.e. frozen) such that well characterized cells are available for administration to the patient. Methods for the production of gene-modified allogeneic cells are described for example in WO 00/72686A1, expressly incorporated by reference herein.

In one approach to preparing genetically modified transgene-expressing allogeneic cells, one or more therapeutic agent-encoding nucleic acid sequences (transgenes) are introduced into a cell line that is an allogeneic tumor cell line (i.e., derived from an individual other than the individual being treated). In another approach, one or more transgenes are introduced into separate allogeneic tumor cell lines. In general, the cell or population of cells is from a tumor cell line of the same type as the tumor or cancer being treated. The tumor and/or tumor cell line may be of any form of cancer, including, but not limited to, carcinoma of the bladder, breast, colon, kidney, liver, lung, ovary, cervix, pancreas, rectum, prostate, stomach, epidermis; a hematopoietic tumor of lymphoid or myeloid lineage; a tumor of mesenchymal origin such as a fibrosarcoma or rhabdomyosarcoma; or another tumor, including a melanoma, teratocarcinoma, neuroblastoma, glioma, adenocarcinoma and non-small lung cell carcinoma.

In one aspect of the invention, an allogeneic tumor cell is modified by introduction of an adenoviral vector having a chimeric fiber protein wherein the chimeric fiber protein comprises at least a portion of a Subgroup C adenovirus shaft region and at least a portion of the head region of a Subgroup B adenovirus adenovirus, wherein the head region binds CD46, e.g., an Ad5 or Ad2 shaft and at least a portion of an Ad35 head which comprises a nucleic acid sequence encoding a transgene, e.g., a cytokine such as GM-CSF, operatively linked to a promoter and expression control sequences necessary for expression thereof. In another aspect, the same allogeneic tumor cell or a second allogeneic tumor cell is modified by introduction of another adenoviral vector having a chimeric fiber protein as described above which comprises at least one additional transgene operatively linked to a promoter and expression control sequences necessary for expression thereof. The nucleic acid sequence encoding the transgene(s) may be introduced into the same or a different allogeneic tumor cell using the same or a different vector. The nucleic acid sequence encoding the transgene(s) may or may not further comprise a selectable marker sequence operatively linked to a promoter. Desirably, the allogeneic cell line expresses high levels of a cytokine, e.g., GM-CSF.

In practicing the invention, one or more allogeneic cell lines are incubated with an autologous cancer antigen, e.g., an autologous tumor cell (which together comprise an allogeneic cell line composition), then the allogeneic cell line composition is administered to the patient. Typically, the cancer antigen is provided by (on) a cell of the cancer to be treated, i.e., an autologous cancer cell. In such cases, the composition is rendered proliferation-incompetent, typically by irradiation, wherein the allogeneic cells and cancer cells are plated in a tissue culture plate and irradiated at room temperature using a Cs source, as detailed above. The ratio of allogeneic cells to autologous cancer cells in a given administration will vary dependent upon the combination.

Any suitable route of administration can be used to introduce an allogeneic cell line composition into the patient, preferably, the composition is administered subcutaneously, intradermally or intratumorally.

The use of allogeneic cell lines in practicing present invention provides the therapeutic advantage that, through administration of a genetically modified transgene-expressing cell line to a patient with cancer, together with an autologous cancer antigen, paracrine production of a therapeutic transgene such as a cytokine results in an effective immune response to a tumor. This obviates the need to culture and transduce autologous tumor cells for each patient.

Bystander

In one further aspect, the present invention provides a universal immunomodulatory genetically modified transgene-expressing bystander cell that expresses at least one transgene. The same universal bystander cell line may express more than one transgene or individual transgenes may be expressed by different universal bystander cell lines. The universal bystander cell line comprises cells which either naturally lack major histocompatibility class I (MHC-I) antigens and major histocompatibility class II (MHC-II) antigens or have been modified so that they lack MHC-I antigens and MHC-II antigens. In one aspect of the invention, a universal bystander cell line is modified by introduction of an adenoviral vector having a chimeric fiber protein wherein the chimeric fiber protein comprises at least a portion of a Subgroup C adenovirus shaft region and at least a portion of the head region of a Subgroup B adenovirus adenovirus, wherein the head region binds CD46, e.g., an Ad5 or Ad2 shaft and at least a portion of an Ad35 head wherein the vector comprises a nucleic acid sequence encoding a transgene, e.g., a cytokine such as GM-CSF, operatively linked to a promoter and expression control sequences necessary for expression thereof. In another aspect, the same universal bystander cell line or a second a universal bystander cell line is modified by introduction of a vector comprising a nucleic acid sequence encoding at least one additional transgene operatively linked to a promoter and expression control sequences necessary for expression thereof. The nucleic acid sequence encoding the transgene(s) may be introduced into the same or a different universal bystander cell line using the same or a different vector. The nucleic acid sequence encoding the transgene(s) may or may not further comprise a selectable marker sequence operatively linked to a promoter. Any combination of transgene(s) that stimulate an anti-tumor immune response finds utility in the practice of the present invention. The universal bystander cell line preferably grows in defined, i.e., serum-free, medium, preferably as a suspension.

An example of a preferred universal bystander cell line is K562 (ATCC CCL-243; Lozzio et al., Blood 45(3): 321-334 (1975); Klein et al., Int. J. Cancer 18: 421-431 (1976)). A detailed description of the generation of human bystander cell lines is described for example in U.S. Pat. No. 6,464,973, expressly incorporated by reference herein.

Desirably, the universal bystander cell line expresses high levels of the cytokine, e.g., GM-CSF.

In practicing the invention, the one or more universal bystander cell lines are incubated with an autologous cancer antigen, e.g., an autologous tumor cell (which together comprise a universal bystander cell line composition), then the universal bystander cell line composition is administered to the patient. Any suitable route of administration can be used to introduce a universal bystander cell line composition into the patient. Preferably, the composition is administered subcutaneously, intradermally or intratumorally.

Typically, the cancer antigen is provided by (on) a cell of the cancer to be treated, i.e., an autologous cancer cell. In such cases, the composition is rendered proliferation-incompetent by irradiation, wherein the bystander cells and cancer cells are plated in a tissue culture plate and irradiated at room temperature using a Cs source, as detailed above.

The ratio of bystander cells to autologous cancer cells in a given administration will vary dependent upon the combination. With respect to GM-CSF-producing bystander cells, the ratio of bystander cells to autologous cancer cells in a given administration should be such that therapeutically effective level of GM-CSF is produced. In addition to the GM-CSF threshold, the ratio of bystander cells to autologous cancer cells should not be greater than 1:1. Appropriate ratios of bystander cells to tumor cells or tumor antigens can be determined using routine methods known in the art.

The use of bystander cell lines in practicing the present invention provides the therapeutic advantage that, through administration of a cytokine-expressing bystander cell line and at least one additional cancer therapeutic agent (expressed by the same or a different cell) to a patient with cancer, together with an autologous cancer antigen, paracrine production of an immunomodulatory cytokine, results in an effective immune response to a tumor. This obviates the need to culture and transduce autologous tumor cells for each patient.

Typically a minimum dose of about 3500 Rads is sufficient to inactivate a cell and render it proliferation-incompetent, although doses up to about 30,000 Rads are acceptable. In some embodiment, the cells are irradiated at a dose of from about 50 to about 200 rads/min or from about 120 to about 140 rads/min prior to administration to the mammal. Typically, when using irradiation, the levels required are 2,500 rads, 5,000 rads, 10,000 rads, 15,000 rads or 20,000 rads. In one embodiment, a dose of about 10,000 Rads is used to inactivate a cell and render it proliferation-incompetent. It is understood that irradiation is but one way to render cells proliferation-incompetent, and that other methods of inactivation which result in cells incapable of multiple rounds of cell division but that retain the ability to express transgenes (e.g. cytokines) are included in the present invention (e.g., treatment with mitomycin C, cycloheximide, and conceptually analogous agents, or incorporation of a suicide gene by the cell).

Compositions and Methods for Practicing the Invention

The invention provides a pharmaceutical composition comprising a recombinant adenoviral vector and/or particle of the invention and/or a tumor cell which has been genetically modified using a recombinant adenoviral vector and/or particle of the invention and a pharmaceutically acceptable carrier, together with methods for using the same.

In one aspect, the invention provides compositions comprising a therapeutically effective amount of an adenoviral vector of the invention in a pharmaceutically acceptable carrier, suitable for local or systemic administration to individuals in unit dosage forms, sterile parenteral solutions or suspensions, sterile non-parenteral solutions or oral solutions or suspensions, oil in water or water in oil emulsions and the like. Formulations for parenteral and non-parenteral drug delivery are known in the art. Compositions also include lyophilized and/or reconstituted forms of the adenoviral vectors and particles of the invention. Acceptable pharmaceutical carriers are, for example, saline solution, protamine sulfate (Elkins-Sinn, Inc., Cherry Hill, N.J.), water, aqueous buffers, such as phosphate buffers and Tris buffers, or Polybrene (Sigma Chemical, St. Louis Mo.) and phosphate-buffered saline and sucrose. The selection of a suitable pharmaceutical carrier is deemed to be apparent to those skilled in the art from the teachings contained herein. These solutions are sterile and generally free of particulate matter other than the desired adenoviral virions. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. Excipients that enhance infection of cells by adenovirus may be included.

In one embodiment, an adenoviral vector of the invention is administered to a host in an amount that is effective to inhibit, prevent, or destroy the growth of tumor cells (i.e., a therapeutically effective amount). This may be accomplished, for example, through replication of the viral vector in the tumor cells and/or expression of a transgene following transduction of the tumor cells. Such administration may occur by any amount that is effective to deliver the adenoviral vector to the tumor target cells, such as by systemic administration or by direct injection of the vector into a tumor. In one approach, the vector is administered systemically in an amount of at least 5×10⁹ viral particles per kilogram body weight and in general, such an amount does not exceed 1×10¹³ viral particles per kilogram body weight. In another approach, the vectors are administered intratumorally in an amount of at least 2×10¹⁰ viral particles and in general such an amount does not exceed 1×10¹³ viral particles per kilogram body weight. In yet another approach, the vectors are instilled into the bladder of the subject. In such cases, the bladder may be pre-treated with a bladder enhancer such as described in US20040131590. The exact dosage to be administered is dependent upon a variety of factors including the age, weight, and sex of the patient, and the size and severity of the tumor being treated. The vectors may be administered one or more times. Single or multiple administrations of the compositions can be carried out with dose levels and pattern being selected by the treating physician. If necessary, the immune response may be diminished by employing a variety of immunosuppressants, or removal of preexisting antibodies, so as to permit repetitive administration and/or enhance replication by reducing the immune response to the viruses. Administration of the adenoviral vectors of the present invention may be combined with other antineoplastic protocols, numerous examples of which are known in the art. Such antineoplastic protocols will vary dependent upon the type of cancer under treatment. Exemplary components of antineoplastic protocols are further described below.

Delivery can be achieved in a variety of ways, employing liposomes, direct injection, systemic injection, catheters, topical applications, inhalation, etc.

It follows that the invention provides a method of treating a subject having a neoplastic condition (typically a patient with cancer), comprising administering a therapeutically effective amount of an adenoviral vector of the invention to the subject. The adenoviral vector may be replication competent or replication defective. One aspect of the present invention relates to the use of genetically modified, transgene-expressing tumor cells which are rendered proliferation incompetent (i.e., by irradiation) and administered to a subject. The genetically modified, proliferation incompetent tumor cells are administered in therapeutically effective amounts to a subject, typically a human cancer patient.

The tumor cells are modified by transduction with an adenoviral vector of the invention as described hereinabove. In one embodiment, the invention utilizes a single transduction of a population of tumor cells by one recombinant adenovirus vector encoding one or more one transgenes. In other embodiments, the invention relies on multiple transduction (infections) of a population of tumor cells with the same recombinant adenovirus vector. In yet other embodiments, the invention utilizes multiple or single transductions by multiple recombinant adenoviruses encoding different transgenes.

Administration of the transduced cells to a mammal results in the regulation, in a stimulatory manner, of the individual's systemic immune response.

In other aspects, the invention relates to the use of modified tumor cells (such as primary tumor cells) expressing one or more transgenes for the reversal or suppression of pre-existing tumors. The tumor cells may also be transduced to express a multi-drug resistance protein (MDR). The MDR may be encoded on the same adenoviral vector, a different adenoviral vector or even a non-adenoviral vector. Other vectors may be used in combination with the adenoviral vectors of the invention to transduce tumor cells. Different vectors may be delivered to the tumor cells at essentially the same time or at other times that are appropriate for the desired effect.

In another aspect, the present invention provides a method of enhancing the immune response to a tumor antigen. In this method, primary tumor cells are modified with adenoviral vectors of the invention to express a transgene and administered to a subject, such as a human cancer patient. The modified tumor cells are administered in a therapeutically effective amount and in such a manner that an increased the immune response of the mammal to the antigen is achieved (e.g. intradermal or subcutaneous).

In a related embodiment, both genetically modified tumor cells and an adenoviral vector of the invention is administered to a subject. Such genetically modified tumor cells may be autologous, allogeneic or bystander cells and administration may take place at the same time or may be sequential.

The cells transduced to express the transgene and the tumor cells harbored by the mammal are not necessarily of the same tumor type. For example, bystander cells may express the transgene in vivo and not be of the same cell or tumor as the tumor harbored by the mammal. In another example, a cell transduced to express GM-CSF may not be of the same tumor type, but may express a tumor antigen in common with the tumor cells of the mammal. Therefore, increasing an immune response to the mammal's tumor.

The tumor and the transduced tumor cells (i.e. primary tumor cells) are typically of the same type of cancer and have common antigens. The cancer may be of any type, including, but not limited to, bladder cancer, bone cancer, brain & spinal cord cancers, brain & spinal cord cancers, cervical cancer, colorectal cancer, endometrial & other uterine cancers, esophageal cancer, gallbladder & bile duct cancers, gastric (stomach) cancer, head & neck cancers, kidney cancer, leukemias, liver cancer, lung cancer, lymphomas, melanoma, multiple myeloma, neuroblastoma, ovarian cancer, pancreatic cancer, prostate cancer, hematologic disorders, retinoblastoma, skin cancer, soft-tissue sarcoma, testicular cancer, thyroid cancer, uterine cancer, Wilms' Tumor, pre-neoplastic lesions, cancerous polyps, etc.

In one embodiment, the method comprises administering a proliferation-incompetent-tumor cell which has not been transduced and an adjuvant e.g., an adenoviral vector of the invention, the adjuvant being administered concomitant with or sequential to, the administration of the cytokine-expressing tumor cell. In one approach the adenoviral vector of the invention comprises the coding sequence for a transgene, e.g., a cytokine such as GM-CSF and is administered concomitant with a proliferation-incompetent tumor cell, wherein the tumor cell is autologous or allogeneic.

Typically the subject is a human patient. For human patients, if a heterologous coding sequence is included in the vector, the heterologous coding sequence may be of human origin although genes of closely related species that exhibit high homology and biologically identical or equivalent function in humans may be used if the product of the heterologous coding sequence does not produce/cause an adverse immune reaction in the recipient. In one embodiment, the heterologous coding sequence codes for a therapeutic protein or therapeutic RNA. A therapeutic active amount of a nucleic acid sequence or a therapeutic gene is an amount effective at dosages and for a period of time necessary to achieve the desired result. This amount may vary according to various factors including but not limited to sex, age, weight of a subject, and the like.

One of ordinary skill will appreciate that, from a medical practitioner's or patient's perspective, virtually any alleviation or prevention of an undesirable symptom (e.g., symptoms related to disease, sensitivity to environmental factors, normal aging, and the like) would be desirable.

The pharmaceutical compositions of this invention may be given following, preceding, in lieu of, or in combination with, other therapies relating to generating an immune response or treating cancer in an individual. For example, the mammal may previously or concurrently be treated by chemotherapy, radiation therapy, and other forms of immunotherapy and adoptive transfer. For example, the compositions of this invention may be used in combination with such chemotherapeutic agents as Cisplatin, combination Cisplatin/Cyclophaphamide, Taxol or Cisplatin/Cyclophosphamide/Doxorobicin. Where such modalities are used, they may be employed in a way or at a time that does not interfere with the immunogenicity of the compositions of this invention. The mammal may also have been administered another vaccine or other composition in order to stimulate an immune response. Such alternative compositions may include tumor antigen vaccines, nucleic acid vaccines encoding tumor antigens, anti-idiotype vaccines, and other types of cellular vaccines, including cytokine-expressing tumor cell lines.

In one embodiment, the transduced cells are processed to remove most additional components used in preparing the cells. In particular, fetal calf serum, bovine serum components, or other biological supplements in the culture medium are removed. In one embodiment, the cells are washed, such as by repeated gentle centrifugation, into a suitable pharmacologically compatible excipient. Compatible excipients include isotonic saline, with or without a physiologically compatible buffer like phosphate or Hepes and nutrients such as dextrose, physiologically compatible ions, or amino acids, and various culture media, particularly those devoid of other immunogenic components. Carrying reagents, such as albumin and blood plasma fractions and nonactive thickening agents, may also be used. In one embodiment, non-active biological components, to the extent that they are present in the pharmacological preparation, are derived from a human, and may even be obtained previously from the subject to be treated.

Yet other aspects of the invention relate to kits for regulating the immune response of a mammal to a tumor antigen. These kits include a recombinant adenovirus of the invent in which has a chimeric fiber protein comprising at least a portion of a Subgroup C adenovirus shaft region and at least a portion of the head region of a Subgroup B adenovirus adenovirus, wherein the head region binds CD46, e.g., an Ad5 or Ad2 shaft and at least a portion of an Ad35 head and may or may not further comprise a nucleic acid encoding a transgene, e.g., a cytokine such as GM-CSF. The kit further includes a container useful, e.g., for placing and containing a tumor cell or tumor fragment, and within which a tumor cell is transduced with the recombinant virus. In some embodiments, the kit further includes a digestive enzyme which converts the tumor tissue to a single cell suspension. In some embodiments, the enzyme is collagenase. In some embodiments, the kit further includes an adjuvant which comprises proliferation-incompetent tumor cells which have not been transduced with the recombinant virus comprising genetic material encoding a cytokine.

### EXAMPLES

The present invention is described by reference to the following examples, which are offered by way of illustration and are not intended to limit the invention in any manner. Standard techniques well known in the art or the techniques specifically described below are utilized. Those skilled in the art will recognize, or be able to ascertain, using no more than routine experimentation, many equivalents to the specific embodiments of the invention described specifically herein. Such equivalents are intended to be encompassed in the scope of the following claims.

### Example 1

### Human Tumor Cell Lines and Cell Culture

Human head and neck cancer lines and human melanoma cell lines used in the examples described herein are listed in Table 1.

TABLE 1
Tumor cell lines

| Cell line | Description | Source/catalog number |
|---|---|---|
| Head and neck | | |
| cancer cell lines | | |
| A-253 | Human, epidermoid | ATCC, HTB-41 |
| | carcinoma | |
| A431 | Human, epidermoid | ATCC, CRL-2592 |
| | carcinoma | |
| FaDU | Human, squamous cell | ATCC, HTB-43 |
| | carcinoma (SQCC) | |
| SCC-9 | Human, tongue, SQCC | ATCC, CRL-1629 |
| SCC-15 | Human, tongue, SQCC | ATCC, CRL-1623 |
| Detroit 562 | Human, Pharyngeal | ATCC, CCL-138 |
| | carcinoma | |
| CAL 27 | Human, Tongue SQCC | ATCC, CRL-2095 |
| RPMI 2650 | Human, nasal septum, | ATCC, CCL-30 |
| | SQCC | |
| Melanoma Cell | | |
| lines | | |
| A375-luc | Human, skin, | CRL-1619 (modified to |
| | malignant melanoma | express luciferase) |
| A2058 | Human, skin, | ATCC, CRL-11147 |
| | malignant melanoma | |
| C32 | Human, skin, | ATCC, CRL-1585 |
| | malignant melanoma | |
| SK-Mel-28 | Human, skin, | ATCC, HTB-72 |
| | malignant melanoma | |
| WM-266-4 | Human, skin, | ATCC, CRL-1676 |
| | malignant melanoma | |
| G-361 | Human, skin, | ATCC, CRL-1424 |
| | malignant melanoma | |

Human head and neck cancer lines and human melanoma cell lines listed in Table 1 were cultured in RPMI 1640 medium containing 10% FBS. Human prostate cancer cell line, PC3M-2AC6, was cultured in RPMI 1640 medium containing 10% FBS. Human prostate cancer cell line, LNCaP (ATCC, CRL-10995) was cultured in RPMI containing 10% FBS, L-glutamine (2 mM), non-essential amino acid (0.1 mM), sodium carbonate (0.075%) and Sodium pyruvate (1 mM). Human prostate cancer cell line, PC-3 (ATCC, CRL-1435), was cultured in RPMI supplemented with 10% FBS and L-glutamine (2 mM).

### Example 2

### Construction of E1-Deficient, GFP Expressing Fiber Chimeric Ad5 Vectors

To generate Ad5 vector based fiber chimeras, first a shuttle plasmid, pAd5LtRt-SmaI is constructed as described below. First, the left end (bp 1-1009 bp) of Ad5 DNA was amplified by PCR using two primers;

Primer 1 (5′-GAATTCTAGGGATAACAGGGTAATCATCATCAATAATATACCTT-3′; SEQ ID NO:17) and Primer 2 (5′-CCCGGGGTGCTCCACATAAATCT-3′; SEQ ID NO:18). The right terminal 580 bp sequences of Ad5 DNA was amplified using a second set of primers designated Primer 3 (5′-AAGCTTTAGGGATAACAGGGTAATCATCATCAATAATATACCTT-3′; SEQ ID NO:19) and Primer 4 (5′-CCCGGGGGAATACATACCCGCAGG-3′; SEQ ID NO:20). The recognition sequence of I-SceI is incorporated into primers 1 and 3. The first PCR product is digested with EcoRI and SmaI and the second PCR product is digested with HindIII and SmaI. The resulting fragments are gel purified and cloned into EcoRI and HindIII sites of pBlueScript (Stratagene, La Jolla, Calif.) by three-way ligation to generate pAd5LtRt-SmaI. The plasmid pFLAdS.CMV5-GFP, which contains the Ad5 GFP vector genome bordered by I-SceI sites, is generated by combining the SmaI-linearized pAd5LtRt-SmaI and the genomic DNA of Ad5.CMV5-GFP (Qbiogene, Carlsbad, Calif.) in E.coli.

The fiber chimeric vectors are generated in several steps. First the plasmid, pFLAd5.CMV5-GFP, is digested with SmaI, and the fragment containing the left and the right terminal fragments (nt. 1 to 3047 and 32652 to 33231, respectively) of Ad5.CMV5-GFP is gel purified and self-ligated to generate pAd5Lt&RtSmaI-CMV5-GFP. The plasmids pFLAd5.CMV5-GFP5T35H, pFLAd5.CMV5-GFP-35F, pFLAd5.CMV5 -GFP-5T3H and pFLAd5.CMV5-GFP-5T3H-RGD, which contain the full-length Ad5 DNA with the GFP coding region replacing the E1 coding region and containing chimeric fiber coding regions are generated by combining SmaI-linearized pAd5Lt&RtSmaI-CMV5-GFP and the genomic DNA of Av1nBg5T35H, Av1nBg35F, Av1nBg-5T3H (Smith et al., 2003), and CRAd:hUPII-E1a-IRES-E1b/Fb5/3LL-RGD, respectively. The gene encoding the shaft portion of the fiber protein in the conditional replication competent adenovirus, CRAd:hUPII-E1a-IRES-E1b/Fb5/3LL-RGD is derived from Ad5, while the knob coding region is obtained from Ad3. The chimeric fiber protein of this virus is further modified by incorporation of RGD ligand at the carboxyl terminus of the protein. To generate fiber chimeric vectors, the full-length plasmids are digested with I-SceI and transfected into PER.C6 cells.

### Example 3

### Construction of E1-Deficient and Human GM-CSF Expressing Fiber Chimeric Ad5 Vectors

To generate these vectors, first a shuttle plasmid, pAd5LtRtSma-CAGhGM is constructed as described below. A gene encoding hGMCSF is PCR amplified from pDR20hGMF using primers

| | |
|---|---|
| PSR 3 (5-CTTCGAGGAATTCAGGATGTGGCTGCAGAGCCTGCTG-3; | SEQ ID NO:21) |
| and | |
| PSR 4 (5-CTTCGAGAAGCTTACTCCTGGACTGGCTCCCAG-3;. | SEQ ID NO:22) |

Restriction enzyme recognition sequences are incorporated into the primers PSR 3 (EcoRI) and PSR 4 (HindIII) to facilitate the cloning. The PCR product (435 bp) is digested with EcoRI and HindIII and the resulting fragment is gel purified. SV40 late poly(A) signal is PCR amplified from pCAT-basic using primers PSR 6 (5-CTTCGAGAAGCTTCAGACATGATAAGATACATTG-3; SEQ ID NO:23) and PSR 7 (5-CTTCGAGGGATCCTACCACATTTGTAGAGGTTTAC-3; SEQ ID NO:24). Restriction enzyme recognition sequences are engineered into the primers PSR 6 (HindIII) and PSR 7 (BamHI). The PCR product (222 bp) is digested with HindIII and BamHI and the resulting fragment is gel purified. The two gel-purified fragments are cloned into the EcoRI and BamHI sites of pGEM-7Z (Promega Corp, Madison, Wis.) to generate pGMCSF-SV40pA-7Z.

The resulting plasmid is digested with BamHI and the ends are filled with dNTP mix using Klenow fragment. Following phenol-chloroform extraction and ethanol precipitation, the resulting DNA is digested with EcoRI and a 670 bp fragment is gel-purified. Next, pAd5Lt&RtSmaI-CMV5-GFP is digested with BglII, the resulting ends are filled with dNTP mix using Klenow fragment. A 4.3 KB fragment containing the left end (357 bp) and the right end (583 bp) of Ad5 DNA and the plasmid backbone are gel purified. A 1.6 KB fragment containing CAG promoter (CMV enhancer, chicken beta actin promoter and intron and rabbit globin intron) is obtained from pRRLsinhCAG.gammapptpre (Shoji et al., 1997. J. Gen Virol. 78:2657-2664; Miyazaki et al., 1989 Gene 79:269-277.urce) by HindIII and BamHI digestion and gel purification. All three gel-purified fragments are ligated to generate a shuttle plasmid, pAd5LtRt5ma-CAGhGM. Combining the SmaI linearized pAd5LtRtSma-CAGhGM with the genomic DNA of Ad5-CMV5-GFP (see SEQ ID NO:1) in E.coli generated the full-length plasmid, pFLAd5-CAG-hGM-5F. To generate similar full-length clones containing knob-encoding regions derived from Ad35 and Ad3, a new shuttle plasmid, pAd5Lt&RtStu-CAG-hGM is constructed from pFLAd5-CAG-hGM-5F by digestion with StuI followed by gel purification of the restriction enzyme fragment containing the left and right terminal fragments and self-ligation. The resulting plasmid is linearized with StuI, combined with genomic DNA derived from Ad5-CMV5-GFP-5T35H and Ad5-CMV5-GFP-5T3H-RGD in E.coli to generate pFLAd5.CAG-hGM-5T35 and pFLAd5.CAG-hGM-5T3H-RGD, respectively. The full-length plasmids are digested with I-SceI and transfected into PER.C6 cells to generate Ad5.CAG-hGM, Ad5.CAG-hGM-5T35H and Ad5.CAG-hGM-5T3H-RGD.

### Example 4

### A Method for the Preparation of Primary Tumor Cells

Tumor tissue (e.g. lung) obtained surgically is submitted for pathological evaluation to confirm its identity as tumor tissue (i.e. non-small cell lung carcinoma). The remaining tumor is shipped/transported at 40° C. in transport medium (Td medium; alpha-MEM containing 1% human serum albumin) to the lab. In most cases, transporting the tumor is done in less than 24 hours. Immediately after arrival, transportation medium is aspirated and the tumor is washed with 50 ml Td medium and processed to a single cell suspension by mechanical digestion for 30 minutes using a Stomacher laboratory blender (Brinkmann, Westbury, N.Y.). The cell debris and clumps are removed by filtration and Ficol density gradient centrifugation (Amersham Pharmacia, Uppsala, Sweden). The cells are plated in 6-well tissue culture dishes.

### Example 5

### Infection of Cell Lines or Primary Tumor Cells Isolated using the Procedure in Example 3 and Determination of Percentage Transduction

The plated primary tumor cells or cell lines are exposed to Ad5 based GFP vectors (e.g. with chimeric fibers) at one or different multiplicities of infection (MOIs) in medium containing 2% fetal bovine serum (FBS) for 2 hr at 37° C. The serum concentration is increased to 4-10% and the cells are further incubated for 24 hr to allow for GFP expression. The cells are washed with PBS and percentage transduction determined by flow cytometry.

To determine and compare the transduction efficiencies of Ad5 fiber chimeras with that of Ad5 carrying wild type fiber, selected human cell lines are transduced with the vectors at 50 particles per cell (ppc) for two hours at 37° C. in a total volume of 0.5 ml of culture medium containing 2% FBS. The infection medium is replaced with 2 ml of the appropriate culture medium containing 4% FBS. The cells are incubated for 24 hours to allow for transgene (beta-galactosidase or green fluorescent protein, GFP) expression. To monitor beta-galactosidase expression, the cell monolayers are fixed and stained with 5-bromo-4-choloro-3-indolyl-beta-D-galactopyranoside (X-Gal) for 24 h. The percentage of transduction is determined by counting the number of transduced blue cells per total cells in a high power field with a light microscope; three fields per well are counted and average percentage transduction is determined. To monitor the GFP expression, the cell monolayer is detached by trypsin treatment, and then the cells are washed with PBS. The percentage of transduction is determined by flow cytometry. Each vector transduction experiment is carried out in triplicate wells and the average percentage of transduction is determined.

### Example 6

### In vitro Ad5 and Chimeric Fiber Vector Mediated Transduction of Human Cells

Cells were transduced at 50 particles per cell as described in Example 5. Infected cells were monitored for beta-galactosidase expression at 24 hours post-infection. The results are shown in Table 2 and reflect the percentage of transduced cells.

TABLE 2
In vitro Ad5 and Ad5 fiber chimeric vectors mediated transduction
of human cells.

| | | Av1nbg | -5T35H | -35F | -5T3H |
|---|---|---|---|---|---|
| Lung | MRC-5 | 2 | 31 | 14 | 16 |
| | H1299 | 63 | 85 | 55 | 83 |
| | H460 | 16 | 60 | 15 | 17 |
| | DMS 114 | 22 | 44 | 31 | 6 |
| | H446 | 72 | 95 | 54 | 15 |
| | A549 | 22 | 74 | 42 | 43 |
| Prostate | PC3 | 1 | 35 | 13 | 17 |
| | DU145 | 16 | 43 | 28 | 27 |
| | LNCaP | 4 | 7 | 5 | 1 |
| Head | SCC9 | 5 | 22 | 10 | 9 |
| & Neck | Fadu | 0.5 | 15 | 7 | 9 |
| | Detroit 562 | 0.25 | 6 | 2 | 3 |
| | A253 | 0.3 | 3 | 1 | 1 |
| Bladder | SW780 | 9 | 36 | 18 | 17 |
| | RT4 | 4 | 24 | 13 | 10 |
| | UC14 | 4 | 22 | 6 | 7 |
| Kidney | SW839 | 3 | 21 | 8 | 4 |
| | 786-0 | 1 | 28 | 12 | 14 |

### Example 7

### In vitro Ad5 and Chimeric Fiber Vector Mediated Transduction of Primary Human Non-Small Cell Lung Carcinoma Cells

Cells were transduced with fiber chimeric vectors and percentage cell transduction determined by monitoring GFP expression at 24 hr post-infection by FACS as described in example 5. The results are shown in Table 3.

TABLE 3
In vitro Ad5 and Ad5 fiber chimeric vectors mediated transduction
of primary human non-small cell lung carcinoma cells.

| Vector | % Transduction |
|---|---|
| Ad5.CMV5-GFP | 5.8 |
| Ad5.CMV5-GFP-35F | 8.4 |
| Ad5.CMV5-GFP-5T35H | 10.6 |
| Ad5.CMV5-GFP-5T3H | 7.9 |
| Ad5.CMV5-GFP-5T3H- | 8.05 |
| RGD | |

### Example 8

### Density of Select Cell-Surface Receptors in Human Tumor Cell Lines

As described above, melanoma and head and neck cancer (HNC) cell lines are relatively less susceptible to Ad5 infection compared to fiber chimeric adenoviral vectors. To investigate the biological basis of the relative resistance of these cell lines to Ad5 but not to fiber chimeric vectors, cellular levels of receptors used by adenoviral vectors were determined. Cultured tumor cells were washed with PBS and detached from the plate with 0.025% trypsin, washed once with and resuspended in PBS (pH 7.4). The cells were incubated with mouse antibody directed against coxsackie-adenovirus receptor (CAR, Rmcb, Upstate biotechnology, Lake placid, N.Y.), CD46 (Clone E4.3, BD Biosciences, Pharmingen, San Diego, Calif.) αᵥβ₃ (Chemicon International, Temecula, Calif.) or αᵥβ₅ (Chemicon International, Temecula, Calif.) for 30 min at 4° C. Subsequently, the cells were washed three times with PBS and incubated with FITC-conjugated secondary anti-mouse IgG (BD Biosciences, Pharmingen, San Diego, Calif.) for 30 min at 4° C. After washing with PBS, cells were suspended in PBS and analyzed by flow cytometry to determine percentage positive cells. Data for human head and neck cancer cell lines and melanoma cell lines are provided in Tables 4 and 5, respectively.

TABLE 4
Selected cell-surface receptor expression in human head and neck
cancer cell lines (% positive cells)

| | | | | | Detroit |
|---|---|---|---|---|---|
| Virus | A-253 | A431 | FaDu | SCC-9 | 562 |
| CAR | 16 | 22 | 2 | 6 | 3 |
| CD46 | 79 | 64 | 94 | 95 | 93 |

TABLE 5
Selected cell-surface receptor expression in human melanoma cell
lines (% positive cells)

| Virus | WM-266-4 | A375-luc | SK-MEL-28 | G361 | A2058 |
|---|---|---|---|---|---|
| CAR | 0.3 | 2 | 9 | 2 | 39 |
| CD46 | 14 | 69 | 5 | 4 | 25 |
| αvβ3 | 62 | 44 | 32 | 1 | 39 |
| αvβ5 | 2 | 28 | 2 | 1 | 3 |

These studies demonstrate that melanoma and HNC cell lines express low levels of CAR. In contrast, the levels of CD46 detected were relatively high, particularly for head and neck cancer cell lines. In addition, all five tested head and neck cancer cell lines had very low levels of αᵥβ₃ and αᵥβ₅ integrins and therefore could not be detected by flow cytometry; however, the expression levels of αᵥβ₃ integrins in a majority of melanoma cells were high. Thus, the relative susceptibility to fiber chimeric vectors and resistance to Ad5 is likely explained by high expression levels of CD46, the primary receptor for Ad3 and Ad35, and low level of CAR expression, the primary receptor for Ad5 on melanoma and HNC cells.

### Example 9

### Construction of E1-Containing and Human GM-CSF Expressing Fiber Chimeric Ad5 Vectors

E1-containing oncolytic vectors comprising chimeric fibers were generated in several steps. First, the full-length plasmid, pFLAd5, was constructed by combining the SmaI-linearized pAd5LtRtSmaI and the genomic DNA of Ad5 in E.coli. The resulting shuttle plasmid pFLAd5 comprises the Ad5 genome bordered by I-SceI sites. Next, pFLAd5 was digested with XhoI and the fragments containing the left and right terminal fragments of Ad5 were gel purified and self-ligated to generate pAd5-LtRtXhoI. The entire fiber-coding region was deleted using PCR and a recognition sequence for SwaI was inserted to generate pAd5-LtRtXhodelfiber. Combining XhoI-linearized pAd5LtRtXhodelfiber and the genomic DNA of CG0070 (as described for example in WO2005030261) generated the plasmid pFLAr20pAE2fhGmdelfiber containing the full-length CG0070 DNA minus fiber encoding region. A recombinant plasmid, pFBSE5T35H obtained from Genetic Therapy Inc., (GTI) and containing the gene encoding Ad5 fiber shaft and Ad35 fiber knob was digested with XbaI and EcoRV and the fragment containing the chimeric fiber encoding region was gel-purified using standard techniques. The plasmid, pFLAr20pAE2fGm-5T35H in which Ad5 shaft and Ad35 knob replacing Ad5 fiber-coding region was generated by combing SwaI linearized pFLAr20pAE2fhGmdelfiber with the gel-purified fragments in E.coli. A fiber chimeric oncolytic adenoviral vector, OV1191 was generated by digesting pFLAr20pAE2fGm-5T35H with I-SceI and transfecting into PER.C6 cells.

To generate E1-containing oncolytic vector OV1192, a 3.6-kb EcoRI and KpnI restriction enzyme fragment containing the gene encoding chimeric fiber protein (Ad5 shaft and Ad3 knob) was obtained from genomic DNA of CRAd:hUPII-E1a-IRES-Eib/Fb5/3_{LL}-RGD and cloned into pBlueScript to generate pBlue-5T3H-RGD. Next, a 3.16-kb restriction enzyme fragment spanning the fiber-encoding region was obtained by digesting the pBlue-5T3H-RGD with EagI and KpnI. The gel-purified fragment was combined with SwaI-linearized pFLAr20pAE2fhGmdelfiber in E.coli to generate pFLAr20pAE2fbGM-5T3H-RGD. The resulting plasmid was digested with I-SceI and transfected into PER.C6 cells to generate OV1192.

Three additional E1-containing oncolytic vectors that are similar to CG0070, OV1191 and 1192 in which an extra ATG located upstream of proper E1A ATG is deleted were generated in several steps. First, the left and right terminal KpnI restriction enzyme fragment obtained from pFLAr21pAe2fF were self-ligated to generate pAr21Lt&RtKpn-E2f. A 1.3-kb NheI-KpnI fragment was obtained from a full-length plasmid, pAr21pAE2fe (GTI). In this full-length plasmid an extra ATG upstream of E1A ATG has been deleted. This restriction enzyme fragment was used to replace the corresponding fragment from pAr21LtRt-Kpn-E2f to generate pAr21LtRtKpn-E2fe. Combining KpnI linearized pAr21LtRtKpn-E2fe with the genomic DNAs of CG0070, OV1191 and OV1192 generated three full-length plasmids, pFLAr20pAE2fe-5 fiber, pFLAr20pAE2fe-5T35H, and pFLAr20pAE2fe-5T3H-RGD respectively. Linearization with I-SceI digestion and transfection of pFLAr20pAE2fe-5fiber, pFLAr20pAE2fe-5T35H, and pFLAr20pAE2fe-5T3H-RGD into PER.C6 cells generated OV1193, OV1194 and OV1195 respectively.

In addition, oncolytic adenoviral vectors in which E1A expression is placed under the control of the hTERT promoter were generated. First, to replace the E2F-1 promoter with hTERT promoter, a NheI-KpnI restriction enzyme fragment of pAr21LtRtKpn-E2f was replaced with a 1293-bp Nhe-KpnI fragment derived from pAr6pATrtexE3F. The resulting plasmid, pAr21LtRtKpn-Trtex, was linearized with KpnI and combined with genomic DNA derived from CG0070. OV1191, OV1192 to generate pFLAr20pATrtex-5fiber, pFLAr20pATrtex-5T35H and pFLAr20pATrtex-5T3H-RGD. Linearization with I-SceI enzyme digestion and transfection of pFLAr20pATrtex-5fiber, pFLAr20pATrtex-5T35H and pFLAr20pATrtex-5T3H-RGD into PER.C6 cells generated OV1196, OV1197 and OV1198 respectively.

Other adenoviruses used herein include CV802, which is a wild type Ad5 containing all wild type DNA sequence and used a positive control. Add1312 is a replication-defective vector with a deletion in the E1a gene and was used as a negative control vector.

All E1-containing vectors were purified using two rounds of cesium chloride density gradient centrifugation. Virus particle titers were determined by the spectrophotometric method as described previously (e.g., see Mittereder, et al 1996).

### Example 10

### In vitro Ad5 and Chimeric Fiber Vector Mediated Transduction and Cytotoxicity of Human Cells

The in vitro cytotoxicity of Ad5 and Ad5 chimeric fiber vectors of the present invention was determined by exposing panel of tumor and normal cells to serial dilutions of virus for seven days. Cell viability was measured using an MTS cytotoxicity assay performed according to the manufacturer's instructions (CellTiter 96® AQᵤₑₒᵤₛ Non-Radioactive Cell Proliferation Assay, Promega, Madison, Wis.). Absorbance values are expressed as a percentage of uninfected control and plotted versus vector dose. A sigmoidal dose-response curve was fit to the data and EC₅₀ value calculated for each replicate using GraphPad Prism software, version 3.0. The EC₅₀ value is the dose of vector in particles per cell (PPC) that reduces the maximal light absorbance capacity of an exposed cell culture by 50%.

In vitro cytolytic potential of chimeric fiber oncolytic adenoviral vectors was tested in four representative head and neck cancer and melanoma cell lines. These data are summarized Tables 6 and 7.

TABLE 6
EC₅₀ values for representative head and neck cancer cell lines

| Virus | A-253 | SCC-9 | FaDu | A431 |
|---|---|---|---|---|
| CV802 | 16 | 12 | 72 | 31 |
| OV1193 | 205 | 59 | 323 | 291 |
| OV1194 | 20 | 6 | 23 | 55 |
| OV1195 | 7 | 4 | 34 | 20 |
| OV1191 | 20 | 39 | 23 | 49 |
| OV1192 | 14 | 24 | 60 | 30 |
| OV1196 | 189 | 40 | 206 | 302 |
| OV1197 | 13 | 5 | 9 | 28 |
| OV1198 | 11 | 1 | 25 | 38 |

TABLE 7
EC₅₀ values for representative melanoma cell lines

| Virus | WM-266-4 | A375-luc | G-361 | A2058 |
|---|---|---|---|---|
| CV802 | 667 | 45 | 52 | 16 |
| OV1193 | 882 | 377 | 177 | 29 |
| OV1194 | 13 | 58 | 161 | 30 |
| OV1195 | 9 | 19 | 40 | 23 |
| OV1191 | 17 | 27 | 101 | 21 |
| OV1192 | 25 | 14 | 83 | 34 |
| OV1196 | 1253 | 102 | 88 | 34 |
| OV1197 | 8 | 13 | 23 | 15 |
| OV1198 | 9 | 7 | 18 | 23 |

These data demonstrate the low EC₅₀ values for infection with the fiber chimeric vectors, OV1194, OV1195, OV1197 and OV1198 and corresponding resistance to Ad5 is likely explained by high expression levels of CD46, the primary receptor for Ad3 and Ad35, and low level of CAR expression, the primary receptor for Ad5 on melanoma and HNC cells.

### Example 11

### Determination of Human GM-CSF Levels Expressed by Chimeric Fiber Ad Vectors

To evaluate human GM-CSF expression, cultured tumor cells were infected at 50 virus particles/cell, supernatants were collected 24 and 72 hours post infection and subjected to a commercially available ELISA assay (R&D Systems, Minneapolis, Minn.) to quantitate the total GM-CSF expressed. Cultured cell supernatants were diluted 10-fold to 1000-fold in assay buffer. Data were acquired on a spectrophotometer at 490 nm and the data were analyzed using the SoftMax software package. The standard curve for human GM-CSF typically had an R² value >0.995 and the sensitivity of the assay was typically 7.8 pg/mL. The amount of human GM-CSF expressed for representative chimeric fiber vectors for head and neck cancer cell lines is shown in Table 8A (24 hours) and Table 8B (72 hours) and for melanoma cell lines in Table 9A (24 hours) and Table 9B (72 hours).

TABLE 8A
Human GM-CSF expression (ng/10⁶ cells/day) in representative
head and neck cancer cell lines at 24 hours post-infection.

| Virus | A-253 | A431 | Detroit 562 | FaDu | SCC-9 |
|---|---|---|---|---|---|
| OV1193 | 5 | 10 | 2 | 5 | 7 |
| OV1194 | 132 | 212 | 135 | 809 | 561 |
| OV1195 | 147 | 212 | 107 | 398 | 476 |

TABLE 8B
Human GM-CSF expression (ng/10⁶ cells/day) in representative
head and neck cancer cell lines at 72 hours post-infection.

| Virus | A-253 | A431 | Detroit 562 | FaDu | SCC-9 |
|---|---|---|---|---|---|
| OV1193 | 285 | 81 | 84 | 131 | 154 |
| OV1194 | 659 | 384 | 360 | 1073 | 2011 |
| OV1195 | 468 | 376 | 323 | 1163 | 1469 |

TABLE 9A
Human GM-CSF expression (ng/10⁶ cells/day) in representative
melanoma cell lines at 24 hours post-infection.

| Virus | A375 | WM-266-4 | A2058 | G-361 | SK-MEL-28 |
|---|---|---|---|---|---|
| CG0070 | 4 | 0.4 | 7 | 0.3 | 1.3 |
| OV1194 | 370 | 17 | 68 | 11 | 15 |
| OV1195 | 312 | 21 | 63 | 8 | 14 |

TABLE 9B
Human GM-CSF expression (ng/10⁶ cells/day) in representative
melanoma cell lines at 72 hours post-infection.

| Virus | A375 | WM-266-4 | A2058 | G-361 | SK-MEL-28 |
|---|---|---|---|---|---|
| CG0070 | 76 | 14 | 145 | 25 | 70 |
| OV1194 | 2074 | 188 | 672 | 130 | 146 |
| OV1195 | 1347 | 246 | 302 | 177 | 158 |

The results indicate that a high level of expression of human GM-CSF is obtained at 24 and 72 hours post infection when representative head and neck cancer and melanoma cell lines are infected with fiber chimeric vectors, OV1194 and OV1195.

### Example 12

### In vivo Efficacy of Chimeric Fiber Ad Vectors in Xenograft Tumor Models

The efficacy of E1-containing chimeric fiber Ad vectors was evaluated in nude mice bearing FaDu (head and neck cancer) or A375-luc (melanoma) xenografts. Nude mice (Hsd:Athymic Nude-nu; Simonsen Labratories, Gilroy Calif.) were implanted with FaDu (5×10⁶ cells in 100-μl of HBSS) or A375-luc (2×10⁶ in 100-μl of HBSS) in the right flank. When tumors reached 50-150 mm³, mice were sorted into groups (n=10) and treated four times intra-tumorally with 1×10¹⁰ particles of viral agents or PBS in a 50-μl dose volume. The size of tumors were measured twice weekly in two dimensions, and the tumor volume was calculated as W×(L)²×Π/6. Mean tumor volume for each treatment group ± SE mean was plotted versus days after vector injection.

### Example 13

### One Method for Isolating, Transducing with an Adenovirus of the Invention and Readministering Primary Tumor Cells to a Mammal

Tumor cells are resected from cutaneous and subcutaneous lesions, lymph nodes, and lung, liver, and soft tissue metastases. After resection, the specimens are aseptically transferred to a sterile container and transported to the laboratory.

All subsequent procedures are preferably conducted under aseptic conditions. The tumor tissues are disaggregated by either collagenase treatment or by mechanical dissociation and subsequently grown in culture and/or frozen in DMSO with 50 percent FCS (collagenase treated) or human albumin (mechanical dissociation). The numbers of the primary and secondary autologous tumor cells may be expanded in culture, and a portion or all of the cells are subsequently transduced to produce hGM-CSF using a recombinant adenovirus of the invention encoding hGM-CSF.

Preferably after verification that the transduced cells are sterile, viable and producing cytokine, the transduced tumor cells are irradiated, suspended in normal saline, and injected (both intradermally and subdermally at up to five different sites in volumes of 0.25-1.0 ml) back into the original donors. The amount of cells per injection is typically between 5×10⁵ to 5×10⁸ and in one embodiment are between 4×10⁶ and 5×10⁷ cells. Multiple injections may be given. In one embodiment, the multiple injections are performed on difference days (e.g. at 7 day, 14 day, 21 day, 1 month intervals or combinations thereof). In one embodiment, up to three injections are performed. Injection schedules may depend on transduced cell yield, among other things.

### Example 14

### Another Method for Isolating, Transducing with an Adenovirus of the Invention and Readministering Primary Tumor Cells to a Mammal

A surgical procedure is utilized to obtain tumor tissue (e.g. autologous) for vaccine preparation. Vaccine cell preparation involves a 48 hour manufacturing process. Vaccines are manufactured from metastatic tumors procured from sites including lymph node, lung, pleural effusion, adrenal, thyroid subcutaneous nodule, and paraspinous mass.

Briefly, a tumor specimen is removed from an individual, washed with HBSS buffer and cut into portions, preferably weighing 3 grams or less. Each portion is placed into a separate Petri dish into which 5 ml collagenase/gram of tissue is added. The tumor tissue is minced into 1 to 3 mm3 sections and placed in a Stomacher bag.

Preferably the volume is less than 20 ml. The bag is sealed and placed inside a mechanical homogenator or Stomacher Lab Blender and homogenized until the digestion is complete (usually 30 to 40 minutes, i. e., until the contents of the bag is a thick milky suspension with few, if any, undigested pieces of tissue). The homogenate is then diluted with 20 ml growth media, brought to a final volume of 40 ml, and centrifuged at 2,000 RPM, for 10 minutes at 4° C. The supernatant is removed and the cell pellet and resuspended in 10 ml growth media on ice. These steps are repeated until the entire specimen is digested and washed essentially free of enzymes (e.g., collagenase) and in suspension in growth media. The number and viability of cells in the cell suspension can then be determined with Trypan blue.

The cell suspension may then be concentrated to an appropriate density for transduction via centrifugation at 2,000 rpm for 10 min. at 4° C. The pellet is then resuspended to a final cell density appropriate for transduction (e.g. 10⁷ cells/ml). Some cells may be set aside for Delayed Type Hypersensitivity (DTH) testing, establishment of a cell line, and cryopreservation. The remaining cells are transduced with the recombinant adenovirus, for example encoding human GM-CSF.

The number of plaque forming units (pfu) or viral particles required to perform transduction is determined by multiplying the total number of cells (dead+viable, calculated after tumor digestion). For example, if a multiplicity of infection of 10 is desired the total number of cells is multiplied by 10. The virus is then diluted in culture media to 10 times the final concentration. Then, 0.1 ml of the adenovirus-GM-CSF suspension per ml of a tumor suspension is added to the cell suspension. The recombinant adenovirus may be, for example, a standard first generation E1 and E3 gene deleted replication deficient virus with the human GM-CSF cDNA introduced into the E1 deleted region. Of course, the cytokine cDNA can be introduced in other regions of the viral genome.

The cells are incubated at room temperature (i. e., approximately 23-25° C.) for 1 hour with gentle mixing every 10 minutes. The volume of tumor cell suspension is then doubled by adding the appropriate volume of growth media to this suspension. The cell suspension is transferred to appropriate tissue culture containers and the cells incubated at 37° C.

The tumor cells are transduced overnight at 37° C. with the recombinant adenovirus. After overnight transduction, the cells are washed extensively with HBSS and then irradiated within 24 hours with 10,000 Rads using a gamma irradiator. A small aliquot of cells (up to 10% of the total) may be placed into culture for 48 hours in the presence of medium.

If GM-CSF is the transgene the GM-CSF secretion from transduced cells may be determined by ELISA (for example, using a kit commercially available kit, for example, from R & D Laboratories, Minneapolis, Minn.) to document appropriate target levels of GM-CSF production. In some embodiments of the invention, the target level is at least 20 ng GM-CSF/10⁶ cells/24 hours, is at least 40 ng GM-CSF/10⁶ cells/24 hours, is at least 100 ng GM-CSF/10⁶ cells/24 hours or is over 1000 ng GM-CSF/10⁶ cells/24 hours.

In addition, routine cultures for bacteria and fungus may be performed to insure sterility of the manipulated cells. Cells for vaccination and immunologic evaluation may then be frozen in 10% DMSO/90% fetal calf serum and stored in liquid nitrogen.

An example of a vaccination procedure is as follows. Cells are thawed and washed twice with Hanks Buffered Salt (HBSS) solution. For administration, cells are thawed not more one hour prior to administration are resuspended in a total volume of not more than 1 ml with sterile saline. A vaccination constitutes a single injection, preferably given in a volume of not more than 1 ml total, intradermally with a 23 or 25 gauge needle, into the upper arm or the thigh on an alternating basis. The total volume of each injection depends on the dose level administered.

### DESCRIPTION OF THE SEQUENCES IN THE SEQUENCE LISTING

The Sequence Listing associated with the instant disclosure is hereby incorporated by reference into the instant disclosure. The following is a description of the sequences contained in the Sequence Listing:

SEQ ID NO:1 is a nucleotide sequence encoding an Ad5 fiber protein (Ad5-CMV5-GFP; Bp position: 28338-30083)

SEQ ID NO:2 is an Ad5 fiber amino acid sequence, 581 amino acids in length.

SEQ ID NO:3 is a nucleotide sequence encoding an Ad2 fiber protein

SEQ ID NO:4 is an Ad2 fiber amino acid sequence

SEQ ID NO:5 is a nucleotide sequence encoding an Ad35 fiber protein (Ad5-CMV5-GFP-35F; Bp position 28337-29308)

SEQ ID NO:6 is an Ad35 fiber amino acid sequence, 323 amino acids in length.

SEQ ID NO:7 is the adenovirus fiber consensus motif, KLGXGLXFD/N

SEQ ID NO:8 is the nucleotide sequence of the gene (ORF) encoding 5T35H fiber protein (Ad5-CMV5-GFP-5T35H; Bp position 28338-30110)

SEQ ID NO:9 is the amino acid sequence of 5T35H fiber (the tail and shaft derived from Ad5 and knob region obtained from Ad35): 590 amino acids in length.

SEQ ID NO:10 is the nucleotide sequence of the gene (ORF) encoding 5T3H fiber protein (Ad5-CMV5-GFP-5T3H; Bp position 28338-30097)

SEQ ID NO:11 is the amino acid sequence of 5T3H fiber (the tail and shaft derived from Ad5 and the knob region obtained from Ad3): 587 amino acids in length

SEQ ID NO:12 is the nucleotide sequence of the gene (ORF) encoding 5T3H-RGD fiber protein (Ad5-CMV5-GFP-5T3H-RGD; Bp position 30217-32052)

SEQ ID NO:13 is the amino acid sequence of 5T3H-RGD fiber (the tail and shaft derived from AdS and the knob region obtained from Ad3): 611 amino acids in length. The RGD sequence is located at the carboxyl-terminus of the fiber protein.

SEQ ID NO:14 is an amino acid sequence of a hGM-CSF

SEQ ID NO:15 is a nucleotide sequence encoding a hGM-CSF

SEQ ID NO:16 is a nucleotide sequence of a cDNA encoding a hGM-CSF

SEQ ID NO:17 is Adenovirus 5 PCR Primer 1

SEQ ID NO:18 is Adenovirus 5 PCR Primer 2

SEQ ID NO:19 is Adenovirus 5 PCR Primer 3

SEQ ID NO:20 is Adenovirus 5 PCR Primer 4

SEQ ID NO:21 is human GM-CSF PCR primer PSR3

SEQ ID NO:22 is human GM-CSF PCR primer PSR4

SEQ ID NO:23 is SV40 late poly(A) signal PCR primer PSR6

SEQ ID NO:24 is SV40 late poly(A) signal PCR primer PSR7

What is claimed is:

1. A method of expressing a heterologous nucleic acid in a primary tumor cell comprising transducing said primary tumor cell with an adenovirus vector comprising a chimeric fiber protein wherein said chimeric fiber protein comprises at least a portion of a Subgroup C adenovirus shaft region and at least a portion of the head region of a Subgroup B adenovirus, wherein the head region binds CD46.

2. The method of claim 1, wherein said chimeric fiber protein comprises at least a portion of an Ad5 or Ad2 shaft and at least a portion of an Ad35 head.

3. The method of claim 1, wherein the cell is transduced in vitro.

4. The method of claim 1, wherein the cell is transduced in vivo.

5. The method of claim 1, wherein the chimeric fiber protein comprises from about amino acids 46 to 136 of SEQ ID NO:6.

6. The method of claim 1, wherein the chimeric fiber protein comprises from about amino acids 47 to 399 of SEQ ID NO:2 or amino acids 47 to 399 of SEQ ID NO:4.

7. The method of claim 5, wherein the chimeric fiber protein, comprises from about amino acids 47 to 399 of SEQ ID NO:2 or amino acids 47 to 399 of SEQ ID NO:4.

8. The method of claim 1, wherein said primary tumor cell is a cell selected from the group consisting of a lung tumor cell, a prostate tumor cell, a head and neck tumor cell, a bladder tumor cell and a kidney tumor cell.

9. The method of claim 1, wherein said primary tumor cell is a non-small cell lung tumor cell.

10. The method of claim 1, wherein said adenovirus preferentially replicates in said primary tumor cells.

11. The method of claim 10, wherein said adenovirus comprises a heterologous transcriptional regulatory element (TRE) operatively linked to at least one adenoviral coding sequence that is essential for replication.

12. The method of claim 11, wherein said TRE is selected from the group consisting of a cell-specific TRE, a cell-status specific TRE and a tissue-specific TRE.

13. The method of claim 11, wherein said TRE is selected from the group consisting of a PSA TRE, an E2F TRE, a telomerase (TERT) TRE, an urokinase plasminogen activator (uPA) TRE, a probasin TRE, a tyrosinase related protein-2 TRE, a MART-1 TRE, a CRGL2 TRE and a PRL-3 protein tyrosine phosphatase TRE.

14. The method of claim 11, wherein said adenoviral coding sequence is in an adenoviral coding region selected from the group consisting of E1a, E1b, E2a, E2b and E4.

15. The method of claim 10, wherein said adenovirus comprises a deletion of part or all of the E1B 19-kDa region.

16. The method of claim 1, wherein said adenovirus comprises a deletion of an adenoviral coding sequence essential for replication.

17. The method of claim 16, wherein said adenovirus is replication incompetent in the primary tumor cell.

18. The method of claim 17, wherein said adenoviral coding sequence essential for replication is selected from the group consisting of E1a, E1b, E2a, E2b and E4.

19. The method of claim 1, wherein said adenovirus comprises a deletion of at least one adenoviral E3 coding sequence.

20. The method of claim 19, wherein said adenoviral E3 coding sequence is selected from the group consisting the coding sequences for the 19K, 14.7K, 14.5K, 12.5K, 11.6K, 10.4K and 6.7K E3 proteins.

21. The method of claim 19, wherein all of the E3 coding sequences are deleted.

22. The method of claim 19, wherein said adenovirus comprises the 10.4K, 14.5K and 14.7K E3 coding sequences.

23. The method of claim 1, wherein said adenovirus comprises all of the native E3 coding sequences.

24. The method of claim 1, wherein said adenovirus comprises a GM-CSF coding sequence operatively linked to regulatory elements wherein GM-CSF is expressed in said primary tumor cell.

25. The method of claim 24, wherein said GM-CSF coding sequence is a human sequence.

26. The method of claim 24, further comprising inactivating the transduced tumor cell and administering the transduced primary tumor cell to a mammal.

27. The method of claim 26, wherein said tumor cell is autologous for said mammal.

28. The method of claim 26, wherein said tumor cell is allogeneic for said mammal.

29. The method of claim 26, wherein said mammal contains tumor cells of the same type as the transduced tumor cells.

30. The method of claim 29, wherein said mammal is a human.
